(19) United States
(12) Patent Application Publication
(10) Pub. No.: US 2026/0263071 A1
(43) Pub. Date: Sep. 10, 2026

(54) SURGICAL STAPLE CARTRIDGE INSERTION AND PROTECTION DEVICES

(71) Applicant: INTUITIVE SURGICAL OPERATIONS, INC., Sunnyvale, CA (US)

(72) Inventors: Jonathan BORST, Sunnyvale, CA (US); Atal PATEL, Mission Viejo, CA (US)

(73) Assignee: INTUITIVE SURGICAL OPERATIONS, INC., Sunnyvale, CA (US)

(21) Appl. No.: 19/482,122

(22) PCT Filed: Apr. 29, 2024
(86) PCT No.: PCT/US2024/026826

§ 371 Date: Nov. 6, 2025

Related U.S. Application Data
(60) Provisional application No. 63/499,786, filed on May 3, 2023.
(60) Provisional application No. 63/596,472, filed on Nov. 6, 2023.

(51) Int. Cl. A61B 17/072

(52) U.S. Cl. CPC A61B 17/07207

(57) ABSTRACT

A device for inserting a staple cartridge into a surgical stapler instrument that has a first jaw with an anvil and a second jaw with a cavity for receiving the staple cartridge, the first and second jaws being movable between open and closed positions. The staple cartridge insertion device comprises a main body sized for positioning between the first and second jaws of the stapler instrument when the first and second jaws are in the open position and a coupling element for removably attaching the main body to the staple cartridge. The insertion device further comprises at least one alignment feature, e.g., a lateral, vertical, rotational or longitudinal alignment feature, configured to engage at least one of the first and second jaws so as to align the staple cartridge with the first and second jaws during insertion of the staple cartridge. The insertion device may also be employed with a bottom retainer for protecting the staple cartridge and that is removably attached to the insertion device and detachable prior to insertion between the jaws of the surgical instrument. The insertion device may also be employed with a retainer guide block that is removably attached to the insertion device and that helps provide vertical alignment of the insertion device and staple cartridge as they are inserted between the first and second jaws.

### CROSS-REFERENCE TO RELATED APPLICATIONS

This application claims the benefit of U.S. Provisional Application Ser. No. 63/499,786, filed May 3, 2023, and U.S. Provisional Application Ser. No. 63/596,472, filed Nov. 6, 2023, the entire disclosures of which are incorporated herein by reference for all purposes.

### BACKGROUND

The field relates to medical instruments, and more particularly to surgical stapling instruments for use in surgeries.

Minimally invasive medical techniques are intended to reduce the amount of extraneous tissue that is damaged during diagnostic or surgical procedures, thereby reducing patient recovery time, discomfort, and deleterious side effects. One effect of minimally invasive surgery, for example, is reduced post-operative hospital recovery times. The average hospital stay for a standard open surgery is typically significantly longer than the average stay for an analogous minimally invasive surgery (MIS). Thus, increased use of MIS could save millions of dollars in hospital costs each year. While many of the surgeries performed each year in the United States could potentially be performed in a minimally invasive manner, only a portion of the current surgeries uses these advantageous techniques due to limitations in minimally invasive surgical instruments and the additional surgical training involved in mastering them.

Improved surgical instruments such as tissue access, navigation, dissection and sealing instruments have enabled MIS to redefine the field of surgery. These instruments allow surgeries and diagnostic procedures to be performed with reduced trauma to the patient. A common form of minimally invasive surgery is endoscopy, and a common form of endoscopy is laparoscopy, which is minimally invasive inspection and surgery inside the abdominal cavity. In standard laparoscopic surgery, a patient's abdomen is insufflated with gas, and cannula sleeves are passed through small (approximately one-half inch or less) incisions to provide entry ports for laparoscopic instruments.

Laparoscopic surgical instruments generally include an endoscope (e.g., laparoscope) for viewing the surgical field and tools for working at the surgical site. The working tools differ from those used in conventional (open) surgery, for example, in that the working end or end effector of each laparoscopic tool may be separated from its handle by an extension tube (also known as, e.g., an instrument shaft or a main shaft). The end effector can include, for example, a clamp, grasper, scissor, stapler, cautery tool, linear cutter, or needle holder, among others.

To perform surgical procedures, the surgeon passes working tools through cannula sleeves to an internal surgical site and manipulates them from outside the abdomen. The surgeon views the procedure from a monitor that displays an image of the surgical site taken from the endoscope. Similar endoscopic techniques are employed in, for example, arthroscopy, pelviscopy, nephroscopy, cystoscopy, cisternoscopy, sinoscopy, hysteroscopy, urethroscopy, and the like.

Minimally invasive telesurgical robotic systems are being developed to increase a surgeon's dexterity when working within an internal surgical site, as well as to allow a surgeon to operate on a patient from a remote location (outside the sterile field). In a telesurgery system, the surgeon is often provided with an image, e.g., a three dimensional image, of the surgical site at a control console. While viewing, e.g., a three dimensional, image of the surgical site on a suitable viewer or display, the surgeon performs the surgical procedures on the patient by manipulating master input or control devices of the control console, which in turn control the motion of the servo-mechanically operated slave instruments.

The servomechanism used for telesurgery will often accept input from two master controllers (one for each of the surgeon's hands) and may include two or more robotic arms. A surgical instrument is mounted on each of the robotic arms. Operative communication between master controllers and associated robotic arm and instrument assemblies is typically achieved through a control system. The control system typically includes at least one processor that relays input commands from the master controllers to the associated robotic arm and instrument assemblies and back in the case of, for example, force feedback or the like. One example of a robotic surgical system is the DA VINCI™ system commercialized by Intuitive Surgical, Inc. of Sunnyvale, California.

A variety of structural arrangements have been used to support the surgical instrument at the surgical site during robotic surgery. The driven linkage or “slave” is often called a robotic surgical manipulator, and exemplary linkage arrangements for use as a robotic surgical manipulator during minimally invasive robotic surgery are described, for example, in U.S. Pat. Nos. 7,594,912, 6,758,843, 6,702,805, 6,676,669, 6,246,200, 5,855,583, 5,808,665, 5,800,423, 5,445,166, and 5,184,601, the disclosures of which are fully incorporated herein by reference in their entirety for all purposes.

During the surgical procedure, the telesurgical system can provide mechanical actuation and control of a variety of surgical instruments or tools having end effectors that perform various functions for the surgeon, for example, holding or driving a needle, grasping a blood vessel, dissecting tissue, or the like, in response to manipulation of the master input devices. One type of surgical instrument that may be employed is a surgical stapler, which performs the function of clamping, stapling, and cutting tissue. Surgical staplers have been employed in many different surgical procedures. For example, a surgical stapler can be used to resect a cancerous or anomalous tissue from a gastro-intestinal tract. Many known surgical staplers have opposing jaws that clamp tissue and an actuatable knife to cut the clamped tissue.

Surgical staplers typically include an instrument shaft supporting an end effector having two opposing jaws. In one of the jaws, a replaceable staple cartridge may be mounted. An actuation mechanism deploys staples from the staple cartridge in order to staple tissue clamped between the staple cartridge and the opposing jaw of the end effector. Different types of staple cartridges (or reloads) can be used that have different staple lengths suitable for different tissues to be stapled.

The use of replaceable staple cartridges does, however, gives rise to some operational challenges. For example, the use of replaceable, i.e., reloadable, staple cartridges enables a user, e.g., a surgeon or an operating room technician or the like, to remove a used staple cartridge from the stapler jaw (once that staple cartridge has been fired) and to load, e.g., reload, an unused staple cartridge in its place within the stapler jaw in order to perform a subsequent stapling operation. When the user reloads an unused staple cartridge into the stapler jaw in order to perform a subsequent stapling operation, there is the opportunity for the unused staple cartridge to be loaded incorrectly into the jaw of the stapler instrument. If an unused staple cartridge is loaded incorrectly into the jaw of the stapler instrument, the staple cartridge and/or the stapler instrument may become dysfunctional and fail to adequately staple the tissue.

Accordingly, while the new telesurgical systems and devices have proven highly effective and advantageous, still further improvements would be desirable to overcome the challenges with existing instruments. In particular, it would be desirable to provide improved loading devices for replaceable staple cartridges that facilitate the accurate insertion of these staple cartridges into surgical stapling instruments.

### SUMMARY

The following presents a simplified summary of the claimed subject matter in order to provide a basic understanding of some aspects of the claimed subject matter. This summary is not an extensive overview of the claimed subject matter. It is intended to neither identify key or critical elements of the claimed subject matter nor delineate the scope of the claimed subject matter. Its sole purpose is to present some concepts of the claimed subject matter in a simplified form as a prelude to the more detailed description that is presented later.

There is described herein surgical stapling instruments, removable staple cartridges for such instruments and devices for improved loading and reloading of the staple cartridges into the jaws of such instruments.

In one aspect, a device is provided for inserting a staple cartridge into a surgical stapler instrument that has a first jaw that includes an anvil and a second jaw that includes a cavity for receiving the staple cartridge, the first and second jaws being movable between open and closed positions. The device comprises a main body sized for positioning between the first and second jaws of the stapler instrument when the first and second jaws are in the open position. The main body comprises a coupling element for removably attaching the staple cartridge insertion device to the staple cartridge. The device further comprises at least one lateral alignment feature configured to engage at least one of the first and second jaws so as to laterally align the staple cartridge with the first and second jaws during insertion of the staple cartridge.

In an embodiment, the at least one lateral alignment feature may include a pair of lateral alignment walls extending from the main body and configured to engage outer side walls of the second jaw during insertion. Additionally or alternatively, the at least one lateral alignment feature may include a fin extending downwardly from the main body and configured to engage the second jaw during insertion. Still further, the at least one lateral alignment features may include first and second lateral alignment walls extending from the main body and configured to engage outer side walls of the first jaw during insertion. The first and second lateral alignment walls may extend upwardly from the proximal portion.

In various embodiments, the main body comprises a distal portion having a gripping surface and a proximal portion having a contact surface configured for contacting an upper surface of the staple cartridge. The coupling element may be a longitudinal rib extending from a lower surface of the main body and configured for insertion into a central channel of the staple cartridge.

In embodiments, the staple cartridge insertion device may also include one or more longitudinal ribs extending from a lower surface of a proximal portion of the main body. These ribs may be configured to contact lateral outer surfaces of the staple cartridge when the device is coupled to the staple cartridge. In addition, the staple cartridge insertion device may also include a vertical alignment feature on a proximal portion of the main body, the vertical alignment feature being configured to vertically align the proximal end of the staple cartridge so that it can be correctly inserted into a proximal end of its receiving cavity in the first jaw. The vertical alignment feature may include an angled upper surface sloping downwardly in the proximal direction.

In embodiments, the staple cartridge may include a switch extending downwardly from a proximal portion of the staple cartridge, and the second jaw may include a locking member extending in a substantially longitudinal direction. The vertical alignment feature may be configured to ensure that the switch correctly engages the lockout upon insertion of the cartridge into the second jaw.

In various embodiments, the staple cartridge insertion device may also include a distal seating element configured to contact the first jaw as the first jaw is moved from the open position to the closed position to thereby transfer force from the first jaw to a distal end portion of the staple cartridge. The distal seating element may be a topmost surface of an angled upper surface of a vertical alignment feature.

In another aspect, a loading device is provided for inserting a staple cartridge into a surgical stapler instrument having first and second jaws movable between open and closed positions. The first jaw may include an anvil having staple pockets and the second jaw may include a cavity for receiving the staple cartridge. The loading device comprises a main body sized for insertion between the first and second jaws of the stapler instrument in the open position. The main body comprises a coupling element for removably attaching the main body to the staple cartridge. The coupling element further comprises a longitudinal rib extending from a lower surface of the main body and may be configured for removable insertion into a central channel of the staple cartridge.

The loading device may also include a vertical alignment feature on a proximal end portion of the main body. The vertical alignment feature may provide a surface along which the device may slide along the first jaw of the stapling instrument as the device is inserted by a user between the first and second jaws. The vertical alignment feature may be configured to vertically align the proximal end of the staple cartridge as it is inserted into a proximal end of its receiving cavity in the first jaw.

In embodiments, the vertical alignment feature may include an angled upper surface extending downwardly in the proximal direction. Additionally or alternatively, the vertical alignment feature may include a spring structure located at the proximal end of the device, the spring structure biasing the proximal end of the staple cartridge downwardly during insertion.

In some embodiments, the staple cartridge may include a switch extending downwardly from a proximal portion of the staple cartridge, and the second jaw may include a locking member extending in a substantially longitudinal direction. In such an arrangement, the vertical alignment feature may be configured to maintain at least a portion of the switch below the lockout member as the staple cartridge is inserted into the surgical instrument.

The staple cartridge insertion device may also include a distal seating element disposed between the main body and the first jaw of the stapling instrument. The distal seating element may be configured to contact the first jaw as the first jaw is moved from the open position to the closed position to transfer a force from the first jaw to a distal end portion of the staple cartridge. The staple cartridge insertion device may also include at least one lateral alignment feature extending away from the main body and configured to engage an outer wall of the first or second jaw to laterally align the staple cartridge with the first and second jaws during insertion of the staple cartridge.

In another aspect, a loading device is provided for inserting a staple cartridge into a surgical stapler instrument having first and second jaws movable between open and closed positions. The loading device comprises a main body sized for insertion between the first and second jaws of the stapler instrument in the open position. The main body comprises a coupling element for removably attaching the main body to the staple cartridge. The coupling element further comprises a longitudinal rib extending from a lower surface of the main body and configured for insertion into a central channel of the staple cartridge.

The staple cartridge insertion device may also include a distal seating feature positioned between the main body and the first jaw of the stapling instrument. The distal seating feature may be configured to contact the first jaw as the first jaw is moved by a user from the open position towards the closed position so as to transfer a force from the first jaw to a distal end portion of the staple cartridge and thereby fully seat the distal end of the staple cartridge within the second jaw.

The distal seating feature may include a distal seating bump located on an upper surface of the main body. Additionally or alternatively, the distal seating feature may include angled upper surfaces located above an upper surface of the main body. At least a portion of the angled upper surfaces may contact the first jaw as the first jaw is moved by a user from the open position towards the closed position. The upper angled surfaces may slope proximally towards a vertical alignment feature that may be configured to vertically align the proximal end of the staple cartridge as it is inserted into a proximal end of a receiving cavity in the first jaw. The upper angled surfaces may be formed on first and second lateral alignment walls extending upwardly from the main body. The first and second lateral alignment walls may be configured to engage lateral side walls of the first jaw to laterally align the staple cartridge with the first and second jaws during insertion of staple cartridge. The upper angled surfaces may provide a surface along which the device may be slid by a user along the first jaw of the stapling instrument as the user inserts the device between the first and second jaws.

In another aspect, a kit for performing a surgical procedure comprises a staple cartridge configured for insertion into one of the jaws of a surgical stapler instrument and a loading device removably coupled to the staple cartridge.

In embodiments, the loading device may include a main body sized for positioning between the first and second jaws of the stapler instrument when the first and second jaws are in the open position. The loading device may also include at least one lateral alignment feature configured to laterally align the staple cartridge with the first and second jaws during insertion of the staple cartridge. In addition, the loading device may include a vertical alignment feature configured to vertically align a proximal end of the staple cartridge as it is inserted into a proximal end of the cavity in the first jaw. Still further, the loading device may also include a distal seating feature configured to contact the first jaw as the first jaw is moved by a user from the open position towards the closed position so as to transfer a force from the first jaw to a distal end portion of the staple cartridge sufficient to fully seat the distal end of the staple cartridge within the cavity.

In still other embodiments, the kit may include the surgical stapler itself. For example, in one such configuration of the kit, there is provided a surgical stapler instrument having a first jaw that includes an anvil and a second jaw including a cavity, the first and second jaws movable between open and closed positions. The kit may also include at least one staple cartridge reload configured to be inserted into the cavity of the second jaw. In addition, the kit may also include a staple cartridge insertion device removably attached to the staple cartridge reload.

The staple cartridge insertion device may include a main body sized for positioning between the first and second jaws of the stapler instrument when the first and second jaws are in the open position. The staple cartridge insertion device may also include at least one lateral alignment feature configured to laterally align the staple cartridge with the first and second jaws during insertion of the staple cartridge. In addition, the staple cartridge insertion device may include a vertical alignment feature configured to vertically align a proximal end of the staple cartridge as it is inserted into a proximal end of the cavity in the first jaw. Still further, the staple cartridge insertion device may also include a distal seating feature configured to contact the first jaw as the first jaw is moved by a user from the open position towards the closed position so as to transfer a force from the first jaw to a distal end portion of the staple cartridge sufficient to fully seat the distal end of the staple cartridge within the cavity.

According to still further embodiments, there is provided a device for inserting a staple cartridge into a surgical stapler instrument having a first jaw that includes an anvil and a second jaw including a cavity for receiving the staple cartridge, the first and second jaws movable between open and closed positions. In this embodiment, the device may include a main body sized for positioning between the first and second jaws of the stapler instrument when the first and second jaws are in the open position, the main body having a coupling element for removably attaching the main body to the staple cartridge. In addition, the device may include at least one rotational alignment feature configured to engage at least one of the first and second jaws and configured to substantially rotationally align the staple cartridge with the first and second jaws during insertion of the staple cartridge.

In embodiments, the device may also include at least one lateral alignment feature extending from the main body. The at least one lateral alignment feature may include a pair of lateral alignment wall portions extending proximally from the main body and configured to engage outer side walls of the second jaw during insertion. The at least one rotational alignment feature may include a pair of protuberances, each protuberance extending from one of the pair of lateral alignment wall portions. Each protuberance may extend laterally inwardly from one of the pair of lateral alignment wall portions.

In embodiments, the device may also include third and fourth lateral alignment walls extending from the main body and configured to engage outer side walls of the first jaw during insertion. Additionally or alternatively, the device may also include a vertical alignment feature on a proximal portion of the main body, the vertical alignment feature configured to help the proximal end of the staple cartridge to be correctly inserted into a proximal end of its receiving cavity in the first jaw. In embodiments, the staple cartridge may include a switch extending downwardly from a proximal portion of the staple cartridge, and the second jaw may include a locking member extending in a substantially longitudinal direction, and the vertical alignment feature may be configured to ensure that the switch correctly engages the lockout upon insertion.

In embodiments, the device may also include a distal seating element configured to contact the first jaw as the first jaw is moved by a user squeezing the fist and second jaws towards each other from the open position to the closed position to thereby transfer force from the first jaw to a distal end portion of the staple cartridge. Still further, in embodiments, the device may also include a bottom retainer configured to protect an underside surface of the staple cartridge, the bottom retainer being selectively attachable and detachable from the device such that, when attached, the staple cartridge is disposed between the device and the bottom retainer, and when detached, the staple cartridge and the device remain attached to each other and are suitable to be inserted between the first and second jaw.

According to still further embodiments, there is provided a device for inserting a staple cartridge into a surgical stapler instrument having a first jaw that includes an anvil and a second jaw that includes a cavity for receiving the staple cartridge, the first and second jaws movable between open and closed positions. The device may include an insertion component that includes a main body sized for positioning between the first and second jaws of the stapler instrument when the first and second jaws are in the open position, and at least one alignment feature configured to engage at least one of the first and second jaws and configured to assist alignment of the staple cartridge with the first and second jaws during insertion of the staple cartridge. The device may also include a bottom retainer configured to protect an underside surface of the staple cartridge, the bottom retainer being selectively attachable to and detachable from the insertion component such that, when the bottom retainer is attached to the insertion component, the staple cartridge is disposed between the insertion component and the bottom retainer, and when the bottom retainer is detached from the insertion component, the staple cartridge and the insertion component remain attached to each other and are suitable to be inserted between the first and second jaw.

The main body may include a coupling element for removably attaching the main body to the staple cartridge. The at least one alignment feature may include at least one lateral alignment feature configured to engage at least one of the first and second jaws and configured to substantially laterally align the staple cartridge with the first and second jaws during insertion of the staple cartridge. The at least one lateral alignment feature may include at least one wall extending from the main body and configured to engage at least one of the first and second jaws. The at least one alignment feature may include at least one rotational alignment feature configured to engage at least one of the first and second jaws and configured to substantially rotationally align the staple cartridge with the first and second jaws during insertion of the staple cartridge. The at least one rotational alignment feature may include at least one protuberance extending laterally inwardly from a wall of the insertion component. The at least one alignment feature may include a vertical alignment feature on a proximal end portion of the main body, the vertical alignment feature defining a surface along which the insertion component may slide along the first jaw of the stapling instrument as the insertion component and the staple cartridge are inserted between the first and second jaws.

Additionally or alternatively, the insertion component may include a distal seating element disposed between the main body and the first jaw of the stapling instrument. The distal seating element may be configured to contact the first jaw as the first jaw is moved from the open position to the closed position to transfer a force from the first jaw to a distal end portion of the staple cartridge. The force may be sufficient to seat the distal end of the staple cartridge within the second jaw.

According to still further embodiments, there is provided a device for inserting a staple cartridge into a surgical stapler instrument having a first jaw that includes an anvil and a second jaw that includes a cavity for receiving the staple cartridge, the first and second jaws movable between open and closed positions. In such embodiments, the device may include an insertion component that includes a main body sized for positioning between the first and second jaws of the stapler instrument when the first and second jaws are in the open position. The device may also include a retainer guide block selectively attachable to the insertion component. The retainer guide block may include a vertical alignment feature configured to vertically align a proximal end of the staple cartridge as it is inserted into a proximal end of the cavity in the first jaw. The retainer guide block may include a distal seating feature configured to contact the first jaw as the first jaw is moved by a user from the open position towards the closed position so as to transfer a force from the first jaw to a distal end portion of the staple cartridge sufficient to fully seat the distal end of the staple cartridge within the cavity. The distal seating feature may include slots configured to slidingly receive a distal tip of the first jaw. Alternatively, the distal seating feature may include a channel or a lumen configured to slidingly receive a distal tip of the first jaw.

In addition, the insertion component may include at least one additional alignment feature configured to engage at least one of the first and second jaws and configured to assist alignment of the staple cartridge with the first and second jaws during insertion of the staple cartridge. The at least one additional alignment feature may include at least one lateral alignment feature configured to engage at least one of the first and second jaws and configured to substantially laterally align the staple cartridge with the first and second jaws during insertion of the staple cartridge. The at least one lateral alignment feature may include at least one wall extending from the main body and configured to engage at least one of the first and second jaws. Additionally or alternatively, the at least one additional alignment feature may include at least one rotational alignment feature configured to engage at least one of the first and second jaws and configured to substantially rotationally align the staple cartridge with the first and second jaws during insertion of the staple cartridge. The at least one rotational alignment feature may include at least one protuberance extending laterally inwardly from a wall of the insertion component.

In embodiments, the device may also include a bottom retainer configured to protect an underside surface of the staple cartridge. The bottom retainer may be selectively attachable to and detachable from the insertion component. When the bottom retainer is attached to the insertion component, the staple cartridge may be disposed between the insertion component and the bottom retainer. When the bottom retainer is detached from the insertion component, the staple cartridge, the insertion component and the retainer guide block may remain attached to each other and may be suitable to be inserted between the first and second jaw.

According to still further embodiments, there is provided a kit for performing a surgical procedure with a surgical stapler instrument having a first jaw that includes an anvil and a second jaw including a cavity, the first and second jaws movable between open and closed positions. In embodiments, the kit may include a staple cartridge reload configured to be inserted into the cavity of the second jaw and an insertion component removably attached to the staple cartridge reload. The insertion component may have a main body sized for positioning between the first and second jaws of the stapler instrument when the first and second jaws are in the open position. The insertion component may also have at least one alignment feature configured to assist with alignment of the staple cartridge with the first and second jaws during insertion of the staple cartridge. The kit may also include a bottom retainer configured to protect an underside surface of the staple cartridge. The bottom retainer may be selectively attachable to and detachable from the insertion component such that, when the bottom retainer is attached to the insertion component, the staple cartridge may be disposed between the insertion component and the bottom retainer, and when the bottom retainer is detached from the insertion component, the staple cartridge, the insertion component and the retainer guide block may remain attached to each other and are suitable to be inserted between the first and second jaw.

In embodiments, the kit may also include a retainer guide block selectively attachable to the insertion component. The retainer guide block may include a vertical alignment feature configured to vertically align a proximal end of the staple cartridge as it is inserted into a proximal end of the cavity in the first jaw. The at least one alignment feature may include at least one lateral alignment feature configured to engage at least one of the first and second jaws and configured to substantially laterally align the staple cartridge with the first and second jaws during insertion of the staple cartridge. Additionally or alternatively, the at least one alignment feature may include at least one rotational alignment feature configured to engage at least one of the first and second jaws and configured to substantially rotationally align the staple cartridge with the first and second jaws during insertion of the staple cartridge.

According to still further embodiments, there is provided a kit for performing a surgical procedure with a surgical stapler instrument having a first jaw that includes an anvil and a second jaw including a cavity, the first and second jaws movable between open and closed positions. In such embodiments, the kit may include a staple cartridge reload configured to be inserted into the cavity of the second jaw. The kit may also include an insertion component removably attached to the staple cartridge reload. The kit may also include a retainer guide block selectively attachable to the insertion component, and the retainer guide block may include a vertical alignment feature configured to vertically align a proximal end of the staple cartridge as it is inserted into a proximal end of the cavity in the first jaw. Still further, the kit may include a bottom retainer attached to the insertion component and being selectively detachable therefrom so as to enable the insertion component, the staple cartridge and the retainer guide block to be inserted between the first and second jaw. The insertion component may include at least one lateral alignment feature configured to engage at least one of the first and second jaws and configured to substantially laterally align the staple cartridge with the first and second jaws during insertion of the staple cartridge. Additionally, the insertion component may include at least one rotational alignment feature configured to engage at least one of the first and second jaws and configured to substantially rotationally align the staple cartridge with the first and second jaws during insertion of the staple cartridge.

### BRIEF DESCRIPTION OF THE DRAWINGS

The above and other aspects, features, and advantages of the device described herein will become more apparent in light of the following detailed description when taken in conjunction with the accompanying drawings in which:

FIG. 1A is a perspective view of an example surgical instrument, according to various embodiments;

FIG. 1B is a perspective view of illustrative surgical instrument with a robotically controlled backend mechanism, according to various embodiments;

FIG. 1C is a top view of an operating room employing a robotic surgical system, according to various embodiments;

FIG. 2 is a perspective view of the distal end portion of an example surgical instrument with the jaws in the open position, according to various embodiments;

FIG. 3 is an exploded view of a staple cartridge, according to various embodiments;;

FIG. 4 is a perspective view of a staple cartridge, according to various embodiments;;

FIG. 5A is a partial top view of the end effector of a surgical stapling instrument including a lockout assembly having an unfired reload installed, according to various embodiments;

FIG. 5B is a top view of a lockout assembly of FIG. 5A in the unlocked position, according to various embodiments;

FIG. 5C is a top view of a lockout assembly of FIG. 5A in the locked position, according to various embodiments;

FIG. 6 is a front perspective view of a staple cartridge insertion device, according to various embodiments;

FIG. 7 is a rear perspective view of a staple cartridge insertion device, according to various embodiments;

FIG. 8 is a bottom perspective view of a staple cartridge insertion device, according to various embodiments;

FIG. 9 is a side view of the staple cartridge insertion device being squeezed between the jaws by a user, in accordance with various embodiments;

FIG. 10 is a rear perspective view of a staple cartridge insertion device, according to various embodiments;

FIG. 11 is a side view of a staple cartridge insertion device, according to various embodiments;

FIG. 12 is a rear perspective view of a staple cartridge insertion device, according to various embodiments;

FIG. 13 is a front perspective view of a staple cartridge insertion device in between the jaws of a surgical stapler, according to various embodiments;

FIGS. 14A-E are rear perspective view of portions of a staple cartridge insertion device, according to various embodiments;

FIG. 15 is a rear perspective view of the staple cartridge insertion device shown in FIG. 14D, according to various embodiments;

FIG. 16 is a front perspective view of a staple cartridge, according to various embodiments; and

FIG. 17 is a rear perspective view of a second jaw of a surgical stapler, according to various embodiments.

FIGS. 18A-18B are perspective views that illustrate another example of a staple cartridge insertion device, according to various embodiments.

FIGS. 19A-19B are perspective and side cross-sectional views, respectively that illustrate a staple cartridge insertion device having a longitudinal alignment feature, according to various embodiments.

FIGS. 20A-20B are perspective views that illustrate another example of a staple cartridge insertion device and a bottom retainer, according to various embodiments.

FIGS. 21A-20E are various side perspective views that illustrate another example of a staple cartridge insertion device and a bottom retainer along with a retainer guide block for use therewith, according to various embodiments.

FIGS. 22A-22B are various side and perspective views that illustrate how the staple cartridge insertion device, the bottom retainer, and the retainer guide block can be employed, according to various embodiments.

FIG. 23 is a front perspective view of a retainer guide block selectively attached to an insertion device which is also selectively attached to a staple cartridge, according to various embodiments.

FIGS. 24A-24B are perspective views that illustrate another example of a staple cartridge insertion device and a bottom retainer, according to various embodiments.

FIGS. 25A-25E are front views of a staple cartridge insertion device selectively detachable from a staple cartridge via a retention mechanism, according to various embodiments.

FIGS. 26A-26D are front views of a staple cartridge insertion device selectively engageable with a jaw via a downwardly-extending alignment mechanism, according to various embodiments.

FIGS. 27A-27B are perspective views of a staple cartridge insertion device selectively engageable with a staple cartridge via a staple cartridge removal mechanism, according to various embodiments.

FIGS. 28A-28C are perspective, top and side views of a staple cartridge insertion device selectively engageable with a jaw via a proximally-extending alignment mechanism, according to various embodiments.

### DETAILED DESCRIPTION

Particular embodiments are described hereinbelow with reference to the accompanying drawings; however, it is to be understood that the disclosed embodiments are merely exemplary and may be embodied in various forms. Therefore, specific structural and functional details disclosed herein are not to be interpreted as limiting, but merely as a basis for the claims and as a representative basis for teaching one skilled in the art to variously employ this disclosure in virtually any appropriately detailed structure. Well-known functions or constructions are not described in detail so as to avoid obscuring the device described here in any unnecessary detail.

While discussed herein with respect to a linear surgical stapler where staples are sequentially fired, it should be understood that the features described herein imay be readily adapted for use in any type of surgical staplers that employs reloadable cartridges, e.g., minimally invasive (e.g., laparoscopic) instruments and/or instruments used for open surgery. Likewise, it should be understood that the features of the described surgical instruments may be readily adapted for use in other types of surgical instruments that employs reloadable cartridges, whether or not the surgical instrument applies staples.

Additionally, the features of the described surgical stapling instruments may be readily adapted for use in surgical instruments that are activated using any technique within the purview of those skilled in the art, such as, for example, manually activated surgical instruments, powered surgical instruments (e.g., electro-mechanically powered instruments), robotic surgical instruments, and the like.

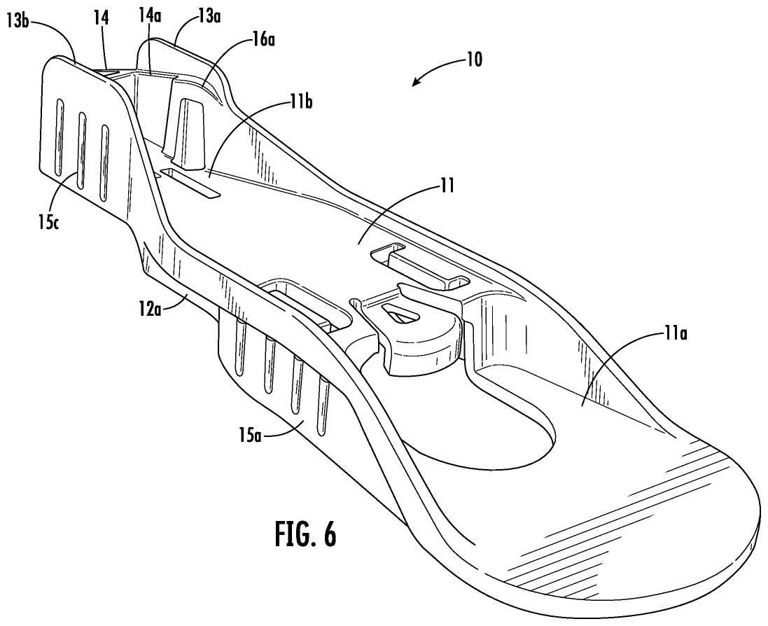

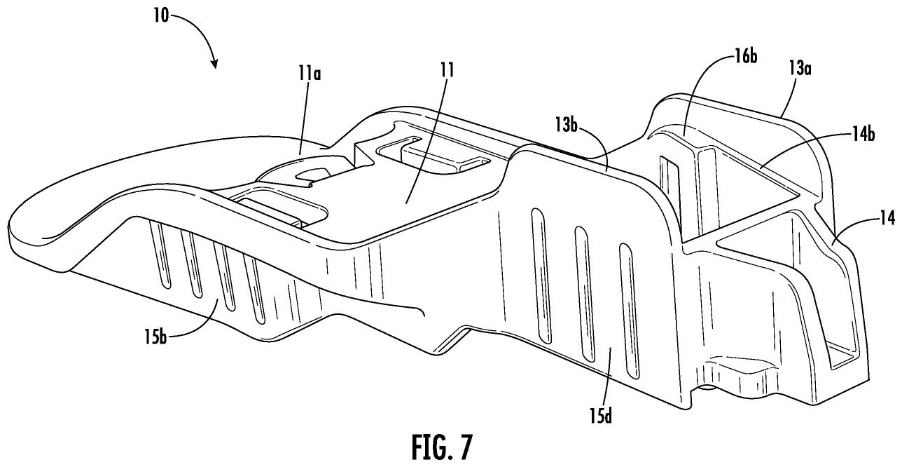

According to various embodiments, there is provided herein a staple cartridge insertion device, such as the staple cartridge insertion device 10, shown by way of example in FIGS. 6-17. The staple cartridge insertion device 10 functions to address certain operational challenges of surgical stapler instruments that employ replaceable staple cartridges, as set forth above. For example, the staple cartridge insertion device 10, according to various embodiments, may help prevent an unused staple cartridge from being loaded incorrectly into the jaw of the stapler instrument which would risk damage or breakage of the stapler components, incorrect or misfiring of the stapler, and/or prevent continued use of the stapler during the surgical procedure.

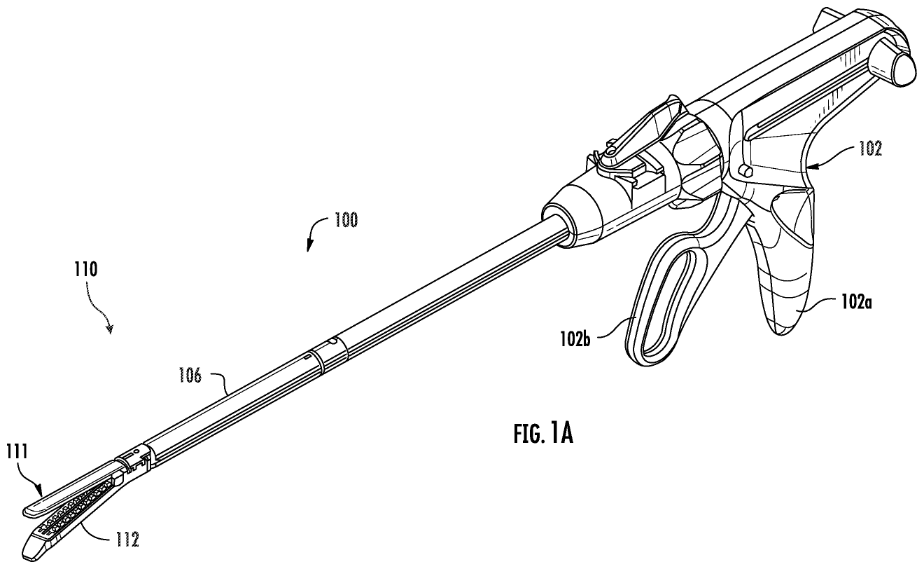

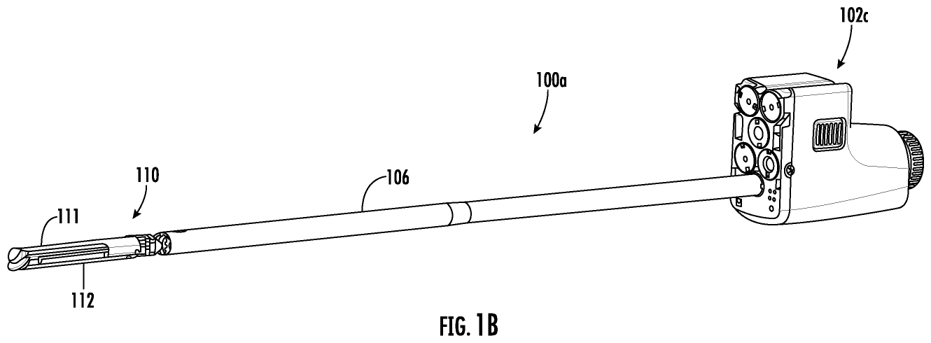

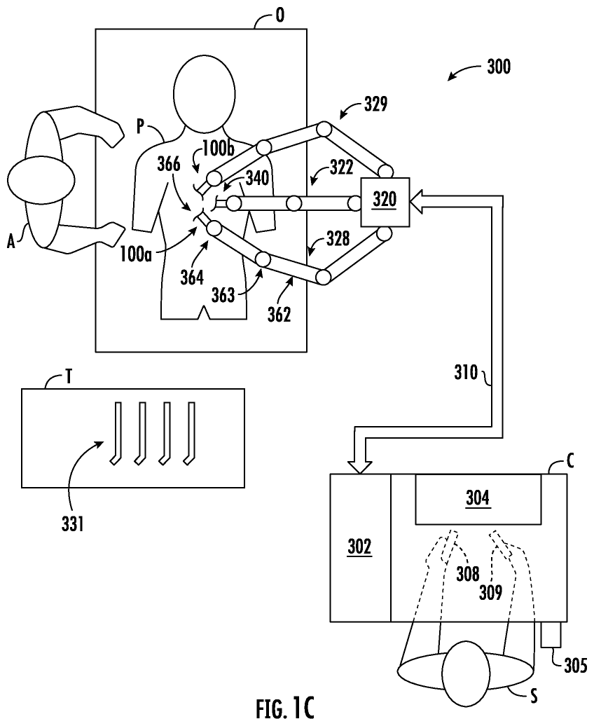

Prior to fully describing the features and operation of the above-reference staple cartridge insertion device 10, it would be beneficial to described the features and operation of an example replaceable staple cartridge, and of an example surgical stapler into which such a replaceable staple cartridge may be reloaded. In this way, the operation by which the staple cartridge insertion device 10 may be employed to reload a replaceable staple cartridge into a surgical stapler can best be understood. FIGS. 1-5C describe such a replaceable staple cartridge, and a surgical stapler into which it may be reloaded, as described more fully in Applicant's co-pending U.S. Provisional Patent Application Ser. No. 63/134,962 entitled “Surgical Stapling Instruments” and filed on Jan. 8, 2021, the entire disclosure of which is hereby fully incorporated by reference herein.

More specifically, FIG. 1A is a perspective view of an example surgical stapler instrument 100 in accordance with various embodiments. The surgical instrument 100, as shown, has a handle assembly 102, and an end effector 110 mounted on an elongated shaft 106. The end effector 110 includes a first, e.g., stationary jaw 111 and a second, e.g., moveable jaw 112. The handle assembly 102 includes a stationary handle 102a and a moveable handle 102b which serves as an actuator for surgical instrument 100.

In certain embodiments, the surgical stapler instrument 100 may instead be operated as part of a robotic surgical system. FIG. 1B illustrates a surgical instrument 100a that includes a robotic backend mechanism 102c instead of the handle assembly shown in FIG. 1A. Backend mechanism 102c typically provides a mechanical coupling between the drive tendons, bands or cables of the instrument and the motorized axes of the mechanical interface of a drive system. Further details of known backend mechanisms and surgical systems are described, for example, in U.S. Pat. Nos. 8,597,280, 7,048,745, and 10,016,244. Each of these patents is hereby incorporated by reference in its entirety.

Still further, in such an arrangement, input couplers may interface with, and be driven by, corresponding output couplers (not shown) of a telesurgical surgery system, such as the system disclosed in U.S Pub. No. 2014/0183244A1, the entire disclosure of which is incorporated by reference herein. The input couplers are drivingly coupled with one or more input members (not shown) that are disposed within the instrument shaft 106. The input members are drivingly coupled with the end effector 110. Suitable input couplers can be adapted to mate with various types of motor packs (not shown), such as the stapler-specific motor packs disclosed in U.S. Pat. No. 8,912,746, or the universal motor packs disclosed in U.S. Pat. No. 8,529,582, the disclosures of both of which are incorporated by reference herein in their entirety. Further details of known input couplers and surgical systems are described, for example, in U.S. Pat. Nos. 8,597,280, 7,048,745, and 10,016,244. Each of these patents is hereby incorporated by reference in its entirety for all purposes.

Actuation mechanisms of surgical instrument 100 may employ drive cables, rods or bands that are used in conjunction with a system of motors and pulleys. Powered surgical systems, including robotic surgical systems that utilize drive cables or bands connected to a system of motors and pulleys for various functions including opening and closing of jaws, as well as for movement and actuation of end effectors are well known. Further details of known drive cable and band surgical systems are described, for example, in U.S. Pat. Nos. 7,666,191, 8,271,230 and 9,050,119 and Publication No. WO 2020/252184, all of which are hereby incorporated by reference in their entireties. While described herein with respect to an instrument configured for use with a robotic surgical system, it should be understood that the actuation and drive assemblies described herein may be incorporated into manually actuated instruments, electro-mechanical powered instruments, or instruments actuated in any other way. In addition, FIG. 1C illustrates still further details of a robotic surgical system in which a surgical stapler instrument may be employed. Additional discussion of FIG. 1C is set forth below.

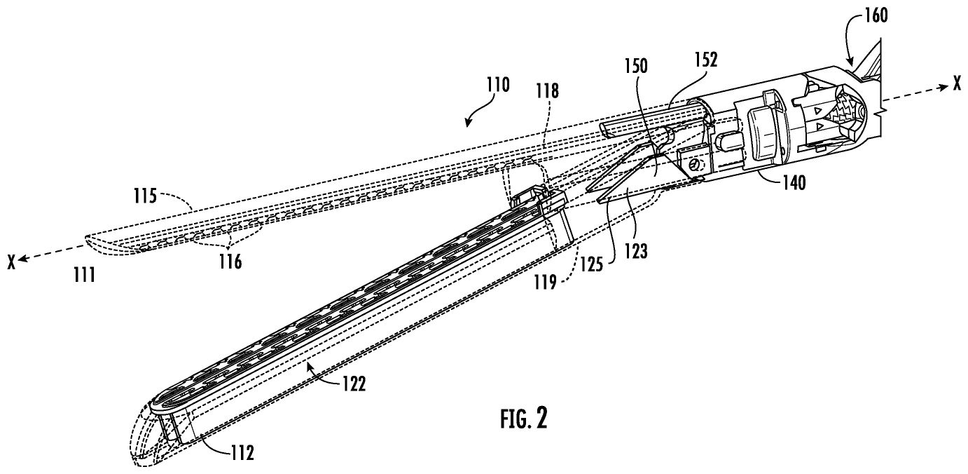

FIG. 2 shows the distal end portion of surgical instrument 100, including an end effector 110 defining a longitudinal axis X-X and having a first jaw 111, a second jaw 112, and a clevis 140 for mounting jaws 111, 112 to the instrument. In certain embodiments, the second jaw 112 is a movable jaw configured to move from an open position to a closed position relative to first jaw 111. In other embodiments, the first jaw 111 may be a movable jaw configured to move between open and closed positions relative to the second jaw 112. In still other embodiments, both jaws 111, 112 may be movable relative to each other. In the exemplary embodiment, the first jaw 112 is a movable jaw 112 configured to move from an open position to a closed position relative to stationary jaw 111. The first jaw 111 includes an anvil 115 having staple-forming pockets 116. In the open position, an unused staple cartridge 122 (sometimes referred to as a fresh or unfired reload) can be loaded into the movable jaw 112 and tissue may be positioned between the jaws 111, 112. In the closed position, the jaws 111, 112 cooperate to clamp tissue such that the staple cartridge 122 and the anvil 115 are in close cooperative alignment.

The surgical instrument 100 may also include a drive member 150 configured to translate distally and retract proximally through the end effector 110. The drive member 150 may have a shuttle 123 integrally formed thereon including an inclined distal portion 125 that sequentially acts on the staple drivers 126 (see, e.g., FIG. 3) upon distal movement of the drive member 150, camming staple drivers 126 (see, e.g., FIG. 3) upwardly, thereby moving the staples 124 (see, e.g., FIG. 3) into deforming contact with the anvil 115. In certain embodiments, the shuttle 123 may be included within the staple cartridge 122 as a separate component. The drive member 150 includes an upper shoe 152 that is substantially aligned with and translates through a channel 118 in the fixed jaw 111, while a lower shoe 154 of the drive member 150 translates through and underneath the jaw 112. Further details of the drive member and actuation will be described below.

As shown in FIG. 3, the staple cartridge 122 may include a plurality of staples 124 supported on corresponding staple drivers 126 provided within respective staple retention openings or pockets 127 formed in the staple cartridge 122. In embodiments, the staple cartridge 122 further includes one or more switches 191 configured to engage a slot 196 formed on the proximal tail 195 of the staple cartridge 122. The functionality of the switches 191 will be described in more detail below.

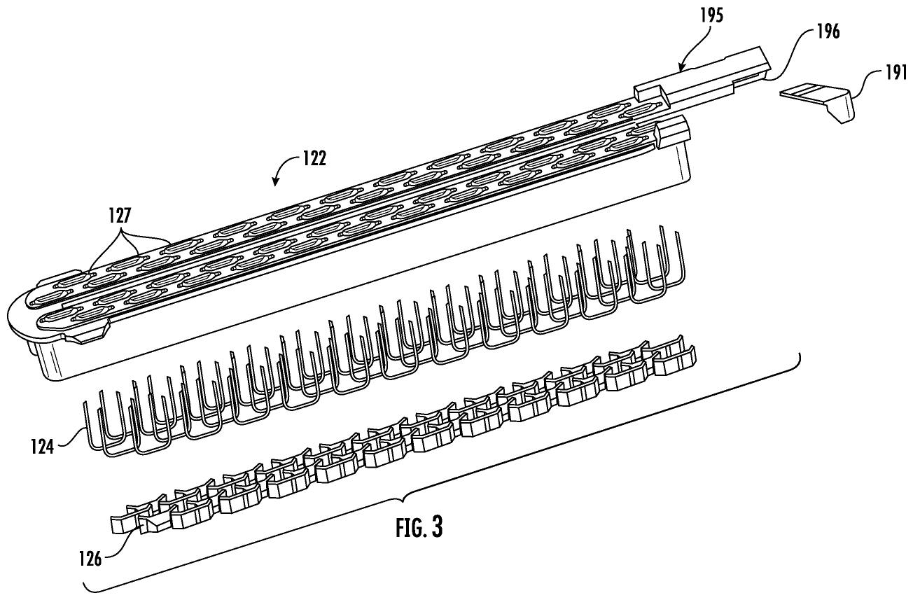

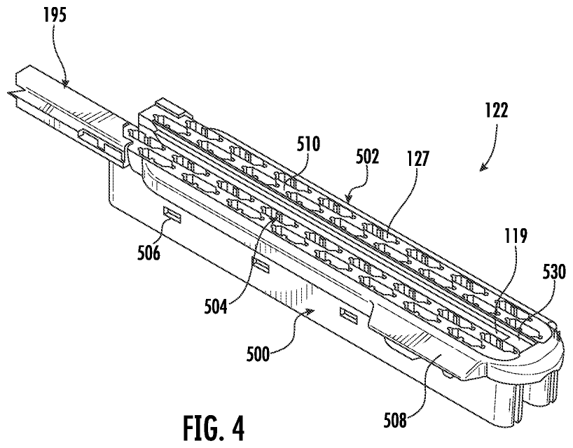

Referring now to FIG. 4, an example embodiment of a staple cartridge 122 will now be described. As shown, the staple cartridge 122 comprises a housing 500 having a central channel 119 for receiving the drive member 150 and first and second staple receiving assemblies 502, 504 extending longitudinally on either side of central channel 119. Each staple receiving assembly 502, 504 comprises at least one linear row of staple pockets 127 for receiving the staples 124. In some embodiments, the staple assemblies 502, 504 comprise two or more substantially parallel, linear rows of staple pockets 127. The staple cartridge 122 may further include one or more openings 506 for cooperating with detents (not shown) in the second jaw 112, and one or more lateral protrusions 508 extending from a distal portion of the housing 500 for cooperating with associated recesses in the jaw 112.

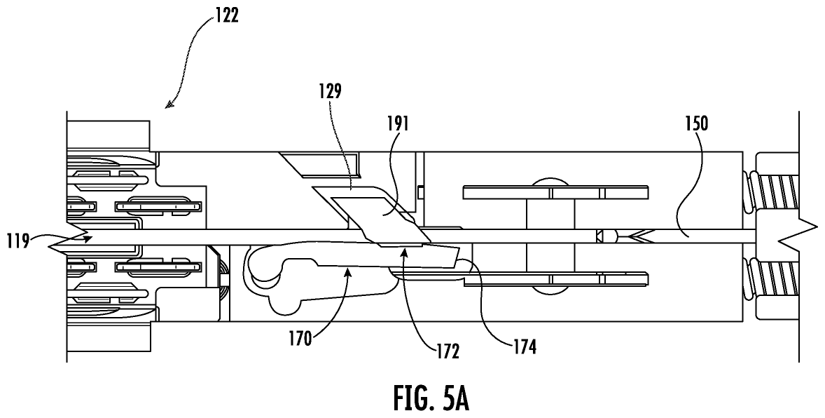

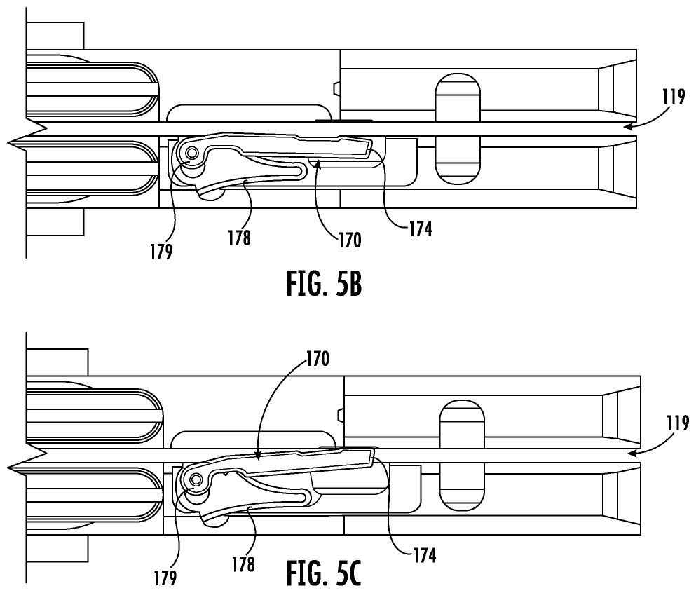

FIG. 5A shows a portion of an example embodiment of the surgical instrument 100 with an unfired staple cartridge (or reload) 122 installed, including portions of the staple cartridge 122, a locking member 170, and the switch 191. When an unfired reload is installed, the switch 191 is in a first home (or default) position. In a fresh, unfired reload, the switch 191 is in contact with a switch engaging portion 172 of the locking member 170, keeping an engagement portion 174 out of the central channel 119. When the locking member 170 is in this disabled position, distal translation of the drive member 150 is permitted, as the locking member 170 will not obstruct movement of the drive member 150 because the engagement portion 174 is held out of alignment with the central channel 119.

FIGS. 5B and 5C show top views of the locking assembly including the locking member 170 in the unlocked or disabled position and the locked position, respectively, with the switch 191 not shown. The locking member 170 pivots about a pivot point 179 that is laterally offset from the central channel 119. The locking member 170 is configured to move in a direction substantially perpendicular to the longitudinal axis of the end effector. A spring 178 biases the engagement portion 174 of the locking member 170 into the central channel 119 to thereby lock the instrument. In the unlocked position of FIG. 5B, the switch 191 (see FIG. 5A) engages the switch engaging portion 172 of the locking member 170, overcoming the bias of the spring 178 and holding the engagement portion 174 out of the central channel 119, thereby permitting distal movement of the drive member 150. When the switch 191 is no longer in contact with the switch engaging portion 172 of the locking member 170, the spring 178 forces the engagement portion 174 of the locking member into the central channel 119 as seen in FIG. 5C, where the engagement portion 174 obstructs distal movement of the drive member 150.

Having now described an example surgical stapler instrument 100 that can be employed in accordance with various embodiments (including manually operated and robotically operated arrangements), there is described hereinbelow in connection with FIGS. 6-17 a staple cartridge insertion device 10. As mentioned above, the staple cartridge insertion device 10 functions to address certain operational challenges of surgical stapler instruments that employ replaceable staple cartridges, for example by helping to prevent an unused staple cartridge from being loaded incorrectly into the jaw of the stapler instrument. By helping to ensure that a replaceable staple cartridge is correctly loaded by a user into its corresponding jaw of a surgical stapler, the staple cartridge insertion device 10, in accordance with various embodiments, may help to prevent potential damage or breakage of the stapler components, may help prevent the surgical stapler from being incorrectly or inadequately fired, and may help enable continued use of the stapler during a surgical procedure.

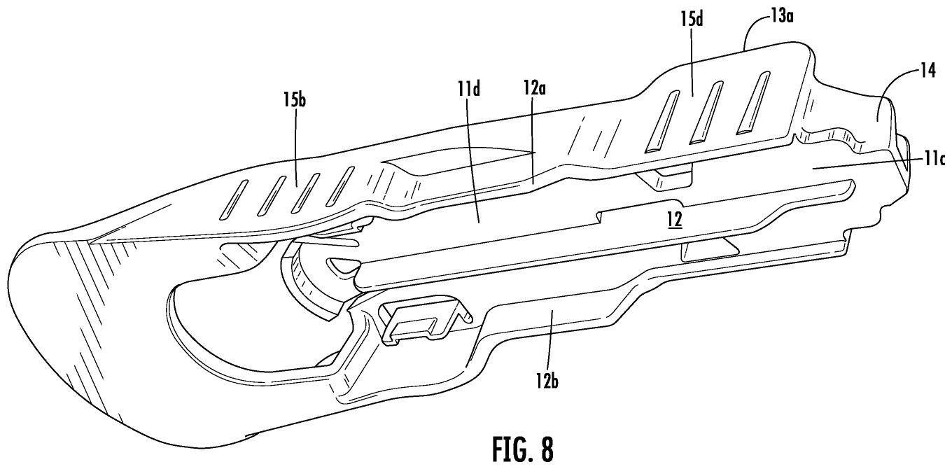

Referring to FIG. 6, there is illustrated an example of a staple cartridge insertion device 10, in accordance with various embodiments. In the embodiment shown, the staple cartridge insertion device 10 may include a main body 11. The main body 11 may include a distal portion 11a and a proximal portion 11b. In addition, as shown in FIG. 8, the main body 11 may include a longitudinal rib 12 that removably couples or attaches the staple cartridge insertion device 10 to a staple cartridge, such as the staple cartridge 122 described above. In this embodiment, the longitudinal rib 12 extends downwardly from a lower surface 11d (again shown in FIG. 8) of the main body 11, and is sized and shaped so as to be removably insertable into the central channel 119 of the staple cartridge 122. It should be recognized that other structures for removably coupling the staple cartridge insertion device 10 to the staple cartridge 122 are contemplated, and that the longitudinal rib 12 shown and described in FIG. 8 is merely one example structure.

The staple cartridge insertion device 10 may also include one or more lateral alignment walls. In the embodiment shown in FIG. 6, the staple cartridge insertion device 10 includes two upwardly-extending lateral alignment walls, specifically upwardly-extending lateral alignment walls 13a and 13b, respectively. As shown, the upwardly-extending lateral alignment walls 13a, 13b extend upwardly from the proximal portion 11b of the main body 11. In addition, the upwardly-extending lateral alignment walls 13a, 13b are sized and shaped so as to extend along and engage the outer side walls of the jaw 111 of the surgical stapler 100 that includes the anvil. The upwardly-extending lateral alignment walls 13a, 13b may have a height of about 0.25 to about 0.30 inches, depending on the dimensions of the anvil jaw 111 and on the space between the first and second jaws 111, 112 of the surgical stapler instrument 100 when the first and second jaws 111, 112 are in the open position.

The staple cartridge insertion device 10 may also include additional lateral alignment walls. In the embodiment shown in FIG. 6, the staple cartridge insertion device 10 includes two downwardly-extending lateral alignment walls or ribs, specifically downwardly-extending lateral alignment walls 12a and 12b, respectively. As shown, the downwardly-extending lateral alignment walls 12a, 12b extend downwardly from a portion of the main body 11 that is longitudinally between its distal portion 11a and its proximal portion 11b. In addition, the downwardly-extending lateral alignment walls 12a, 12b are sized and shaped such that, when the longitudinal rib 12 is inserted into the central channel 119 of the staple cartridge 122, the downwardly-extending lateral alignment walls 12a, 12b extend along the outer side walls of the jaw 112 of the surgical stapler 100 that receives the staple cartridge 122.

In operation, once the staple cartridge insertion device 10 has been removably attached to the staple cartridge 122, e.g., by the longitudinal rib 12 being removably inserted into the central channel 119 of the staple cartridge 122, the upwardly-extending lateral alignment walls 14a, 14b are positioned along the outer sides of the jaw 111, and the staple cartridge insertion device 10 is slid proximally by the user between the jaws 111, 112. As the staple cartridge insertion device 10 is slid proximally by the user between the jaws 111, 112, the upwardly-extending lateral alignment walls 14a, 14b maintain the lateral position of the staple cartridge 122 relative to the jaw 112. Furthermore, the downwardly-extending lateral alignment walls 12a, 12b may also help maintain the lateral position of the staple cartridge 122 relative to the jaw 112 as the staple cartridge insertion device 10 is slid proximally by the user between the jaws 111, 112.

The distal portion 11a of the main body 11 may also include gripping surfaces 15a, 15b, 15c, 15d (best shown in FIGS. 6 and 8), which are configured to be gripped by a user in order to help hold and slide the staple cartridge insertion device 10 relative to the jaws 111, 112. The gripping surfaces 15a and 15b are located relatively nearer to the distal portion 11a of the main body 11 and offer one location at which a user may grip the staple cartridge insertion device 10 with, e.g., a thumb and forefinger, while the gripping surfaces 15c and 15d are located on the proximal portion 11a of the main body 11 and offer another location at which a user may grip the staple cartridge insertion device 10 with such fingers, depending on the user's preference.

In addition, the proximal portion 11b of the main body 11 may include a contact surface 11c, best shown in FIG. 8. The contact surface 11c of the main body 11 may be located on the lower surface of the main body 11, such that the contact surface 11c contacts the upper surface of the staple cartridge 122 when the staple cartridge insertion device 10 is positioned between the jaws 111, 112 of the surgical stapler instrument 100 and as the staple cartridge insertion device 10 is slid proximally by the user between the jaws 111, 112.

In embodiments, the staple cartridge insertion device 10 may also include vertical alignment features (in addition to the lateral alignment features mentioned above) in order to ensure that the staple cartridge 122 is inserted into its corresponding cavity within the jaw 112 at an appropriate relative vertical position. For example, in various embodiments, the staple cartridge insertion device 10 may include a vertical alignment spacer 14 located at the proximal portion 11b of the main body 11. The vertical alignment spacer 14 may extend longitudinally beyond the alignment walls 13a, 13b in the proximal direction. In addition, the vertical alignment spacer 14 may include angled upper surfaces 14a located along the inner surfaces of the alignment walls 13a, 13b. Inserting the staple cartridge 122 into its corresponding cavity within the jaw 112 at an appropriate relative vertical position may help ensure that the switch 191 of the staple cartridge 122 does not inadvertently catch on the lockout features of the jaw 112.

In operation, once the upwardly-extending lateral alignment walls 14a, 14b are positioned along the outer side walls of the jaw 111, the staple cartridge insertion device 10 is slid proximally by the user between the jaws 111, 112 with the vertical alignment features sliding along the surface of the jaw 111 so as to ensure that the structures at the proximal end of the staple cartridge 122, e.g., the switch 191, does not inadvertently get caught on or otherwise incorrectly engage the structures at the proximal ends of the jaws 111, 112, e.g., the lockout features of the jaw 112. In this way, once the staple cartridge insertion device 10 has been fully slid proximally by the user between the jaws 111, 112, the proximal end of the staple cartridge 122 has been correctly seated within the proximal end of its corresponding cavity in jaw 122.

In embodiments, the staple cartridge insertion device 10 may also include distal seating features in order to help ensure that the distal end of the staple cartridge 122 is fully inserted into its corresponding cavity within the jaw 112 prior to a user operating the surgical stapler instrument 100. For example, in various embodiments, the staple cartridge insertion device 10 may include distal seating elements 16a, 16b located at the proximal portion 11b of the main body 11. In the embodiment shown, the distal seating elements 16a, 16b are, e.g., the topmost ridges or bumps located at the highest and most distal ends of the angled upper surfaces 14a, 14b, although other surfaces are contemplated.

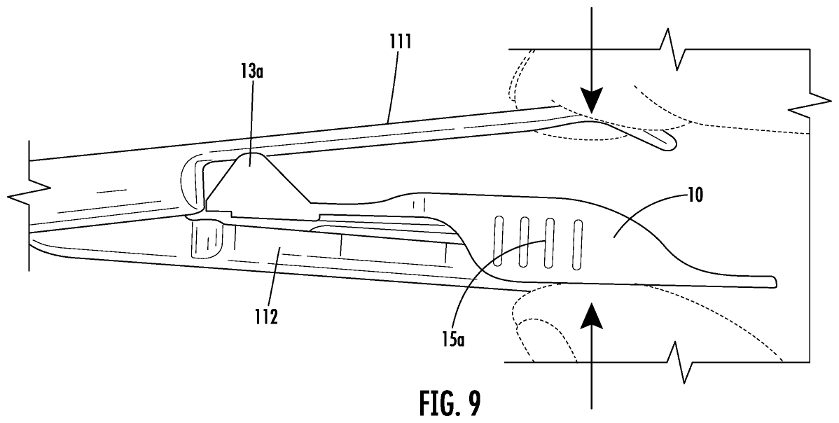

In operation, once the staple cartridge insertion device 10 has been employed to slide the proximal end of the staple cartridge 122 longitudinally between the jaws 111, 112 and into the proximal end of its corresponding cavity within the jaw 112, a user may gently squeeze the first and second jaws towards each other, e.g., by pressing down on the jaw 111 (such as shown in FIG. 9), so as to move the jaw 111, 112 from their open position towards their closed position. In this manner, the jaw 111 transfers a force to the distal seating elements 16a, 16b, which in turn transfers a force to the staple cartridge 122, and in particular to the distal end of the staple cartridge 122 in order to fully seat the distal end of the staple cartridge 122 within the distal end of the cartridge-receiving cavity of jaw 112.

It should be noted that the angled upper surfaces 14a, 14b, although described above as being part of the vertical alignment features, may also help play a role in seating the distal end of the staple cartridge 122, e.g., by also being a surface upon which the jaw 112 may transfer some force to the staple cartridge insertion device 10 and thereby to the distal end of the staple cartridge 122. Of course, other structures or arrangements for the distal seating features are also contemplated, including arrangements in which the distal seating features are not connected to, or otherwise associated with, the vertical alignment features.

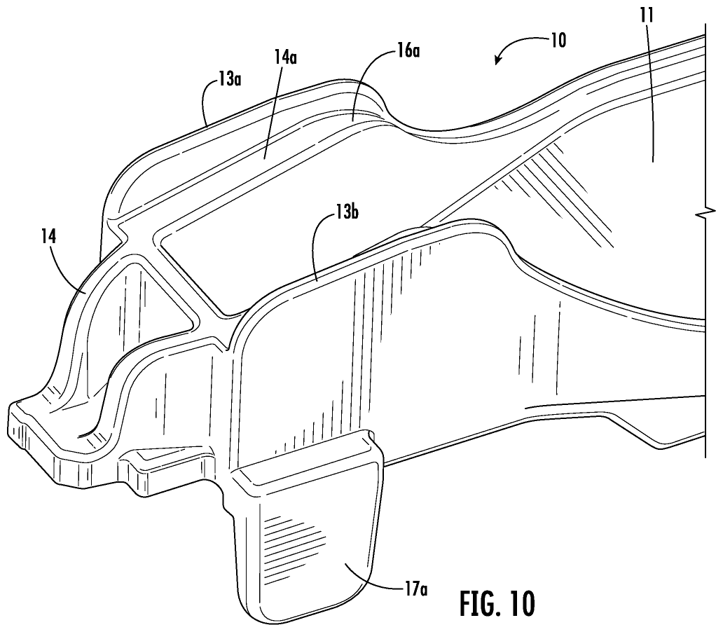

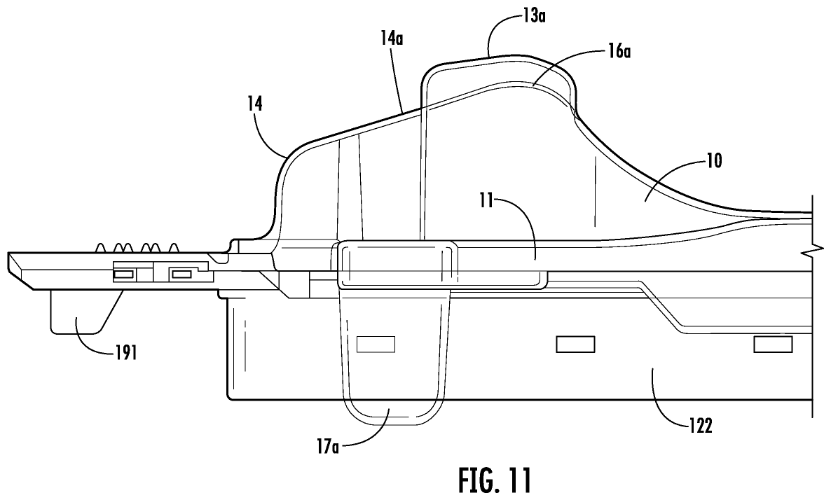

It should be recognized that, in accordance with other embodiments, different or additional structures may be employed in order to provide the functionality described above. For example, while the lateral alignment features described above include, e.g., one or more lateral alignment walls 13a, 13b that engage the outer side walls of the first jaw 111 during insertion of the staple cartridge 122 between the first and second jaws 111, 112 so as to help ensure lateral alignment of the staple cartridge 122 relative to the first and second jaws 111, 112 during such insertion, other structures are also contemplated for such purposes. For example, FIG. 10 illustrates an alternative arrangement in which secondary lateral alignment walls 17a (and 17b located on the opposite side but which is hidden in this view) may be employed, either in addition to (as shown) or instead of, the lateral alignment walls 13a, 13b. In this embodiment, the secondary lateral alignment walls 17a, 17b may engage outer side walls of the jaw 112 (unlike the lateral alignment walls 13a, 13b, which engage the outer side walls of the jaw 111), and may also function to help ensure lateral alignment of the staple cartridge 122 relative to the first and second jaws 111, 112 during such insertion. FIG. 11 is a side view showing a staple cartridge insertion device 10 having such secondary lateral alignment walls 17a, 17b, while being removably attached to a staple cartridge 122. FIG. 13, on the other hand, is a perspective view that illustrates, among other things, the staple cartridge 122 removably attached to the staple cartridge insertion device 10 having such secondary lateral alignment walls 17a, 17b, with both the staple cartridge 122 and the staple cartridge insertion device 10 being positioned between the jaws 111, 112 and the secondary lateral alignment walls 17a, 17b engaging the outer side walls of the jaw 112.

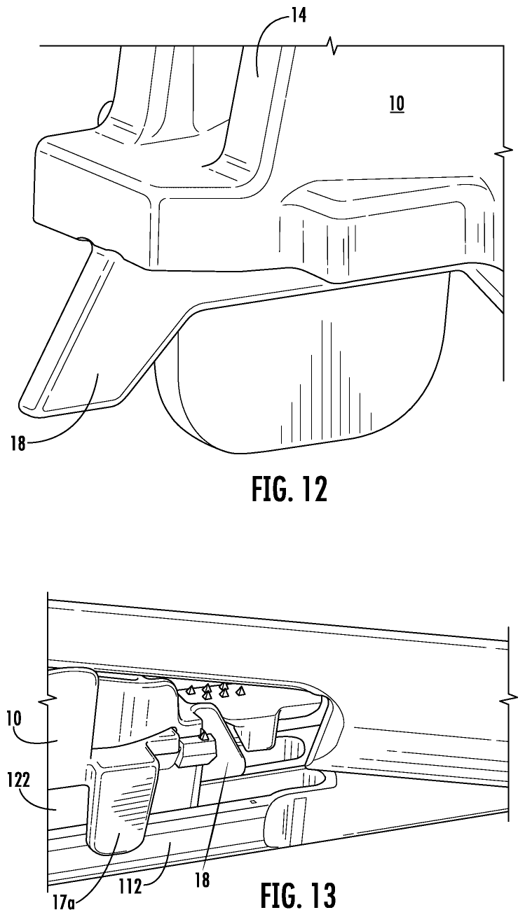

Similarly, the functionality described above in connection with the lateral alignment walls 13a, 13b (and/or the secondary lateral alignment walls 17a, 17b) may additional or alternatively be performed by still other structures. For example, while the lateral alignment features described above include structures that engage the outer side walls of the jaws during insertion of the staple cartridge 122 between the first and second jaws 111, 112 (e.g., the lateral alignment walls 13a, 13b engaging the outer side walls of the first jaw 111, and/or the secondary lateral alignment walls 17a, 17b engaging the outer side walls of the second jaw 112), it is contemplated that the staple cartridge insertion device 10 may include structures that engage other portions of the surgical instrument 100. FIGS. 12 and 13 illustrate an embodiment in which a downward fin 18 is employed to help ensure lateral alignment of the staple cartridge 122 relative to the first and second jaws 111, 112 during insertion. The downward fin 18 does not engage the outer side walls of either of the jaws 1111, 112, but rather engages with structures that are internal to the jaws 111, 112.

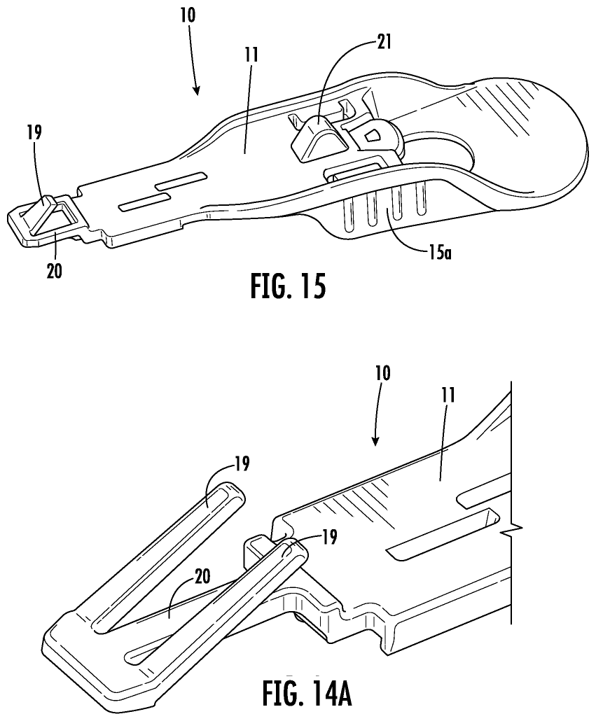

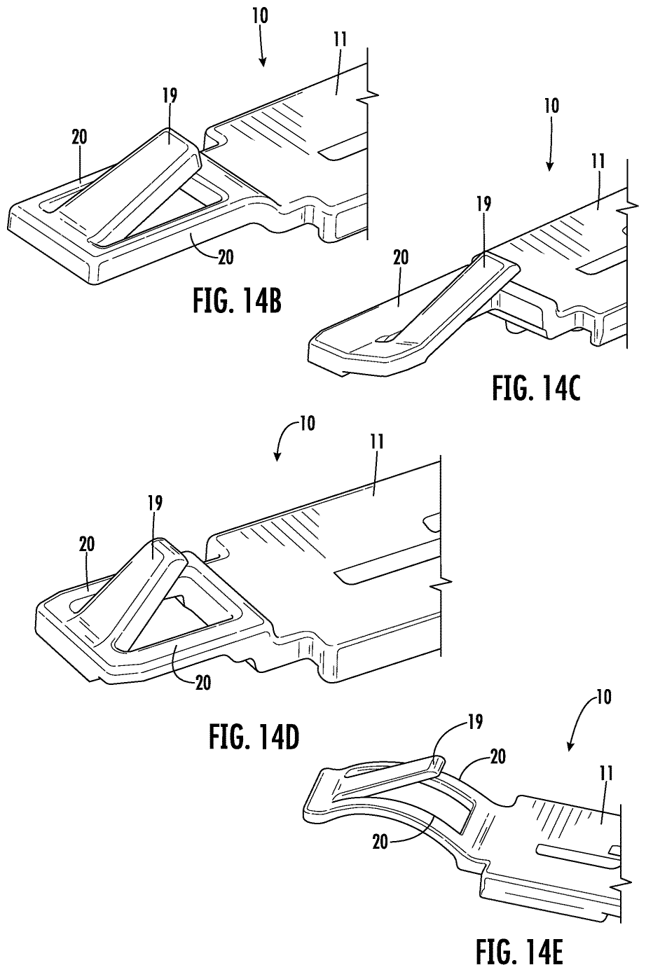

Furthermore, just as there is contemplated different or additional structures that may be employed in order to provide lateral alignment of the staple cartridge 122 relative to the first and second jaws 111, 112 during such insertion, different or additional structures are also contemplated for other purposes. For example, while there is described above various embodiments in which vertical alignment of the staple cartridge 122 is provided by a proximal-extending vertical alignment spacer 14 (extending proximally from the main body 11) and/or a pair of angled upper surfaces 14a, 14b that separately or collectively function to maintain the vertical spacing of, e.g., the switch 191 of the staple cartridge 122 relative to the lockout features of the jaw 112, such vertical alignment may also be achieved by other structures. FIGS. 14A-14E illustrate different embodiments of a vertical alignment feature that may also function to help ensure vertical alignment of the staple cartridge 122 relative to the second jaw 112 while the staple cartridge 122 is being inserted between the first and second jaws 111, 112. Each of the embodiments shown in FIGS. 14A-14E include an extension element 20 that extends proximally relative to the main body 11. In addition, each of the embodiments shown in FIGS. 14A-14E include a spring element 19 that extends at least partially in an upward direction relative to the extension element 20. In this way, the extension elements 20 and the spring elements 19 function as a living hinge that, upon engaging the anvil surface of the first jaw 111 as the staple cartridge insertion device 10 is being inserted, biases the proximal end of the staple cartridge insertion device 10 (and thus the proximal end of the staple cartridge 122) downwardly to help ensure its correct insertion into the proximal end of its receiving cavity within jaw 112.

FIG. 15 illustrates other details of a staple cartridge insertion device 10 having such a living hinge arrangement at its proximal-most end. In this case, FIG. 15 illustrates a full staple cartridge insertion device 10 having the extension element 20 and the spring element 19 shown in FIG. 14D, although the additional details shown and described in FIG. 15 may be employed in any of the embodiments shown in FIGS. 14A-14E, or any other embodiments. In FIG. 15, the staple cartridge insertion device 10 is also illustrated as including a distal seating bump 21 located nearer to the distal end 11a of the main body 11. Similar to the distal seating features described above, the distal seating bump 21 shown in FIG. 14 operates to transfer a force to the distal end of the staple cartridge 122 upon a user squeezing the first and second jaws 111, 112 towards each other from the open position towards a closed position. In this way, the distal seating bump 21 helps ensure that the distal end of the staple cartridge 122 is fully seated within the distal end of its receiving cavity in jaw 112 prior to its being used to staple tissue.

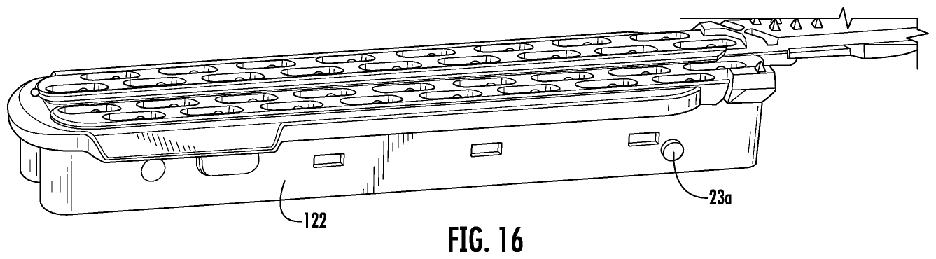

Furthermore, while there are described above various embodiments in which vertical alignment of the staple cartridge 122 is provided by structures located on the staple cartridge insertion device 10, such vertical alignment of the staple cartridge 122 may also be provided by structures that are not located on the staple cartridge insertion device 10. For example, in various embodiments, the vertical alignment of the staple cartridge 122 may be provided by structures located on the staple cartridge 122 itself. FIGS. 16-17 illustrate an example embodiment in which a vertical alignment feature of the staple cartridge 122 is provided by structures located on the staple cartridge 122 itself. As shown in FIG. 16, in this embodiment, the vertical alignment features include a pair of bosses 23a (and 23b which is hidden in this view but which is disposed on the opposite side of the staple cartridge 122) that extend laterally from the outer side wall of the staple cartridge 122. In addition, as shown in FIG. 17, in this embodiment, the vertical alignment features may also include a pair of slots 24a (and 24b which is hidden in this view but which is disposed on the opposite side of the jaw 112) that are recessed within an inner wall of the jaw 112. In operation, when the staple cartridge 122 is inserted between the first and second jaws 111, 112, the pair of bosses 23a, 23b on the outer side walls of the staple cartridge 122 mate with the pair of slots 24a, 24b on the inner walls of the jaw 112 so as to guide the proximal end of the staple cartridge 122 into the proximal end of its receiving cavity within the jaw 112. Of course, it should be recognized that various other structures may be employed on the staple cartridge 122 and/or the jaw 112 so as to provide similar vertical alignment functionality, e.g., the bosses 23a, 23b or slots 24a, 24b may have other shapes or sizes, and/or there may be a different number of bosses 23a, 23b or slots 24a, 24b, and/or the bosses 23a, 23b and slots 24a, 24b may be reversed such that the bosses 23a, 23b are disposed on the jaw 112 and the slots 24a, 24b are disposed on the staple cartridge 122. Of course, various other structures are also contemplated.

Having now described an example surgical stapler instrument 100 that can be employed in accordance with various embodiments (including manually operated and robotically operated arrangements), there is described hereinbelow in connection with FIGS. 6-17 a staple cartridge insertion device 10. As mentioned above, the staple cartridge insertion device 10 functions to address certain operational challenges of surgical stapler instruments that employ replaceable staple cartridges, for example by helping to prevent an unused staple cartridge from being loaded incorrectly into the jaw of the stapler instrument. By helping to ensure that a replaceable staple cartridge is correctly loaded by a user into its corresponding jaw of a surgical stapler, the staple cartridge insertion device 10, in accordance with various embodiments, may help to prevent potential damage or breakage of the stapler components, may help prevent the surgical stapler from being incorrectly or inadequately fired, and may help enable continued use of the stapler during a surgical procedure.

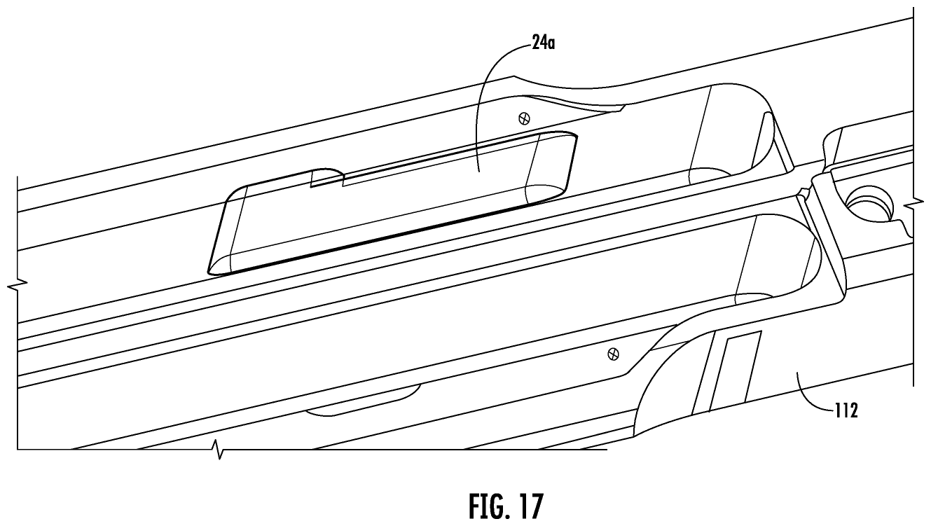

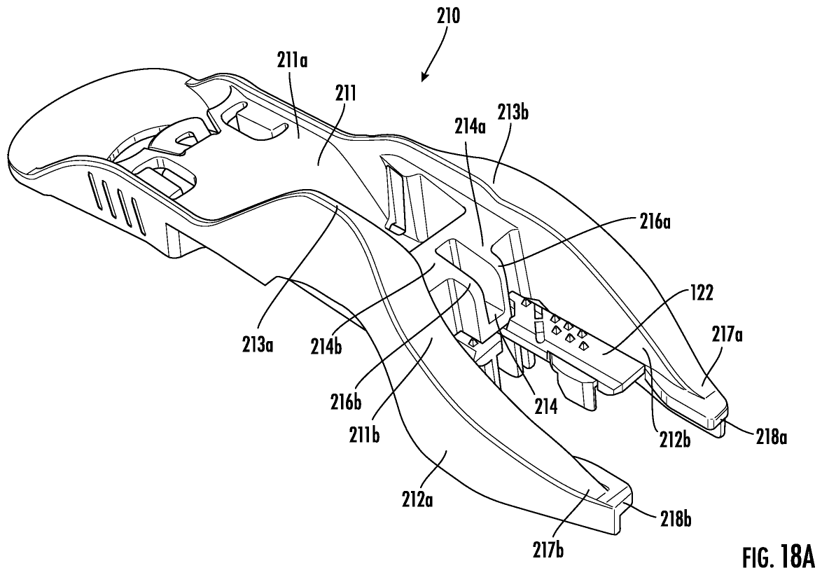

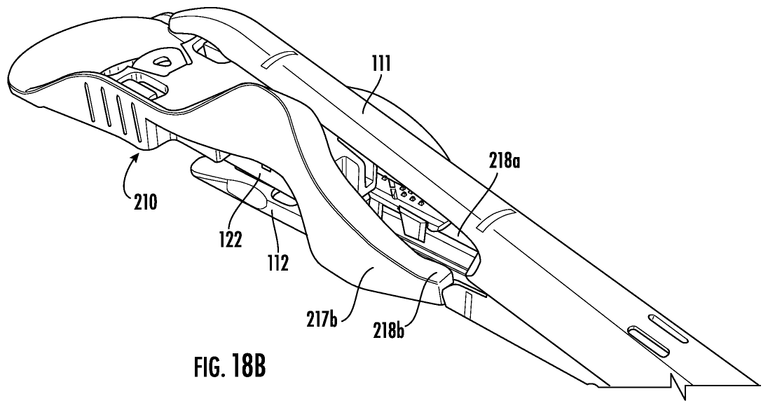

Still other embodiments of a staple cartridge insertion device are contemplated. For example, referring to FIGS. 18A-20B, there is illustrated another example of a staple cartridge insertion device 210, in accordance with various embodiments. In the embodiment shown, the staple cartridge insertion device 210 may include a main body 211. The main body 211 may include a distal portion 211a and a proximal portion 211b. In addition, the main body 211 may include a longitudinal rib (not shown, but similar to the longitudinal rib 12 shown in FIG. 8) that removably couples or attaches the staple cartridge insertion device 10 to a staple cartridge, such as the staple cartridge 122 described above. In this embodiment, the longitudinal rib would extend downwardly from a lower surface (similar to that shown in FIG. 8) of the main body 211, and may be sized and shaped so as to be removably insertable into the central channel 119 of the staple cartridge 122. It should be recognized that other structures for removably coupling the staple cartridge insertion device 210 to the staple cartridge 122 are contemplated, and that a longitudinal rib similar to that shown and described in FIG. 8 is merely one example structure.

The staple cartridge insertion device 210 may also include one or more lateral alignment walls. In the embodiment shown in FIG. 18A, the staple cartridge insertion device 210 includes two upwardly-extending lateral alignment walls, specifically upwardly-extending lateral alignment walls 213a and 213b, respectively. As shown, the upwardly-extending lateral alignment walls 213a, 213b extend upwardly from the main body 211 and are sized and shaped so as to extend along and engage the outer side walls of the jaw 111 of the surgical stapler 100 that includes the anvil. The upwardly-extending lateral alignment walls 213 a, 213 b may have a height of about 0.2 to about 0.30 inches, and preferably about 0.235 inches, depending on the dimensions of the anvil jaw 111 and on the space between the first and second jaws 111, 112 of the surgical stapler instrument 100 when the first and second jaws 111, 112 are in the open position.

The staple cartridge insertion device 210 may also include additional lateral alignment walls. In the embodiment shown in FIG. 18A, the staple cartridge insertion device 210 includes two downwardly-extending lateral alignment walls or ribs, specifically downwardly-extending lateral alignment walls 212a and 212b, respectively. As shown, the downwardly-extending lateral alignment walls 212a, 212b extend downwardly from a proximal portion 211b of the main body 211 and are sized and shaped such that, when the longitudinal rib (such as shown in FIG. 8) is inserted into the central channel 119 of the staple cartridge 122, the downwardly-extending lateral alignment walls 212a, 212b extend along the outer side walls of the jaw 112 of the surgical stapler 100 that receives the staple cartridge 122.

In operation, once the staple cartridge insertion device 210 has been removably attached to the staple cartridge 122, e.g., by the longitudinal rib being removably inserted into the central channel 119 of the staple cartridge 122, the upwardly-extending lateral alignment walls 213a, 213b are positioned along the outer sides of the jaw 111, and the staple cartridge insertion device 210 is slid proximally by the user between the jaws 111, 112. As the staple cartridge insertion device 210 is slid proximally by the user between the jaws 111, 112, the upwardly-extending lateral alignment walls 213a, 213b maintain the lateral position of the staple cartridge 122 relative to the jaw 112. Furthermore, the downwardly-extending lateral alignment walls 212a, 212b also help maintain the lateral position of the staple cartridge 122 relative to the jaw 112 as the staple cartridge insertion device 210 is slid proximally by the user between the jaws 111, 112. Although not shown in FIG. 18A, the staple cartridge insertion device 210 may also include gripping surfaces, such as the gripping surfaces 15a, 15b, 15c, 15d shown in FIGS. 6 and 8, that are configured to be gripped by a user in order to help hold and slide the staple cartridge insertion device 10 relative to the jaws 111, 112. In addition, the main body 211 may include a contact surface, similar to the contact surface 11c shown in FIG. 8, which contacts the upper surface of the staple cartridge 122 when the staple cartridge insertion device 210 is positioned between the jaws 111, 112 of the surgical stapler instrument 100 and as the staple cartridge insertion device 210 is slid proximally by the user between the jaws 111, 112.

In embodiments, the staple cartridge insertion device 210 may also include vertical alignment features in order to ensure that the staple cartridge 122 is inserted into its corresponding cavity within the jaw 112 at an appropriate relative vertical position. For example, in various embodiments, the staple cartridge insertion device 210 may include a vertical alignment spacer 214 located near the proximal portion 211b of the main body 211. In the embodiment shown in FIG. 18A, the vertical alignment spacer 214 may extend longitudinally beyond the upwardly-extending alignment walls 213a, 213b in the proximal direction, while the downwardly-extending lateral alignment walls 212a, 212b extend proximally beyond the vertical alignment spacer 214. In addition, the vertical alignment spacer 214 may include angled upper surfaces 214a, 214b. Inserting the staple cartridge 122 into its corresponding cavity within the jaw 112 at an appropriate relative vertical position may help ensure that the switch 191 of the staple cartridge 122 correctly engages the lockout features of the jaw 112.

In operation, once the upwardly-extending lateral alignment walls 213a, 213b are positioned along the outer side walls of the jaw 111, the staple cartridge insertion device 210 is slid proximally by the user between the jaws 111, 112 with the vertical alignment features sliding along the surface of the jaw 111 so as to ensure that the structures at the proximal end of the staple cartridge 122, e.g., the switch 191, does not inadvertently get caught on or otherwise incorrectly engage the structures at the proximal ends of the jaws 111, 112, e.g., the lockout features of the jaw 112. In this way, once the staple cartridge insertion device 210 has been fully slid proximally by the user between the jaws 111, 112, the proximal end of the staple cartridge 122 has been correctly seated within the proximal end of its corresponding cavity in jaw 122.

In embodiments, the staple cartridge insertion device 210 may also include distal seating features in order to help ensure that the distal end of the staple cartridge 122 is fully inserted into its corresponding cavity within the jaw 112 prior to a user operating the surgical stapler instrument 100. For example, in various embodiments, the staple cartridge insertion device 210 may include distal seating elements 216a, 216b located near the proximal portion 211b of the main body 211. In the embodiment shown, the distal seating elements 216a, 216b are, e.g., the topmost ridges or bumps located at the highest and most distal ends of the angled upper surfaces 214a, 214b, although other surfaces are contemplated.

In operation, once the staple cartridge insertion device 210 has been employed to slide the proximal end of the staple cartridge 122 longitudinally between the jaws 111, 112 and into the proximal end of its corresponding cavity within the jaw 112, a user may gently squeeze the first and second jaws towards each other, e.g., by pressing down on the jaw 111 (such as shown in FIG. 9), so as to move the jaws 111, 112 from their open position towards their closed position. In this manner, the jaw 111 transfers a force to the distal seating elements 216a, 216b, which in turn transfers a force to the staple cartridge 122, and in particular to the distal end of the staple cartridge 122 in order to fully seat the distal end of the staple cartridge 122 within the distal end of the cartridge-receiving cavity of jaw 112.

It should be noted that the angled upper surfaces 214a, 214b, although described above as being part of the vertical alignment features, may also help play a role in seating the distal end of the staple cartridge 122, e.g., by also being a surface upon which the jaw 112 may transfer some force to the staple cartridge insertion device 10 and thereby to the distal end of the staple cartridge 122. Of course, other structures or arrangements for the distal seating features are also contemplated, including arrangements in which the distal seating features are not connected to, or otherwise associated with, the vertical alignment features.

It should be recognized that, in accordance with other embodiments, different or additional structures may be employed in order to provide the functionality described above. For example, while the lateral alignment features described above include, e.g., one or more lateral alignment walls 213a, 213b that engage the outer side walls of the first jaw 111 during insertion of the staple cartridge 122 between the first and second jaws 111, 112 so as to help ensure lateral alignment of the staple cartridge 122 relative to the first and second jaws 111, 112 during such insertion, other structures are also contemplated for such purposes. For example, FIG. 18A illustrates proximally-extending lateral alignment wall portions 217a and 217b of the downwardly-extending lateral walls 212a, 212b. In this embodiment, the proximally-extending lateral alignment wall portions 217a, 217b may engage more proximal regions of the outer side walls of the jaw 112 (unlike the upwardly-extending lateral alignment walls 213a, 213b, which engage the outer side walls of the jaw 111), and may also function to help ensure lateral alignment of the staple cartridge 122 relative to the first and second jaws 111, 112 during such insertion. FIG. 18B, for example, is a perspective view that illustrates, among other things, the staple cartridge 122 removably attached to the staple cartridge insertion device 210 at least in part by the proximally-extending lateral alignment wall portions 217a, 217b, with both the staple cartridge 122 and the staple cartridge insertion device 210 being positioned between the jaws 111, 112 and the proximally-extending lateral alignment wall portions 217a, 217b engaging the outer side walls of the jaw 112.

Still further, in addition to lateral and vertical alignment features, it is also contemplated that the staple cartridge insertion device may include additional alignment features. For example, in embodiments, the staple cartridge insertion device 210 may include rotation alignment features in order to help ensure that the distal end of the staple cartridge 122 is prevented from being inserted into its corresponding cavity within the jaw 112 in a rotated position relative thereto. For example, as shown in FIGS. 18A and 18B, the staple cartridge insertion device 210 may include rotational alignment elements 218a, 218b in the form of tabs or other protuberances that extend laterally inwardly near the proximal-most ends of the proximally-extending lateral alignment wall portions 217a, 217b. In this embodiment, the rotational alignment elements 218a, 218b engage a channel of the jaw 112 during insertion of the staple cartridge 122 between the first and second jaws 111, 112 so as to help ensure rotational alignment of the staple cartridge 122 relative to the first and second jaws 111, 112 during such insertion.

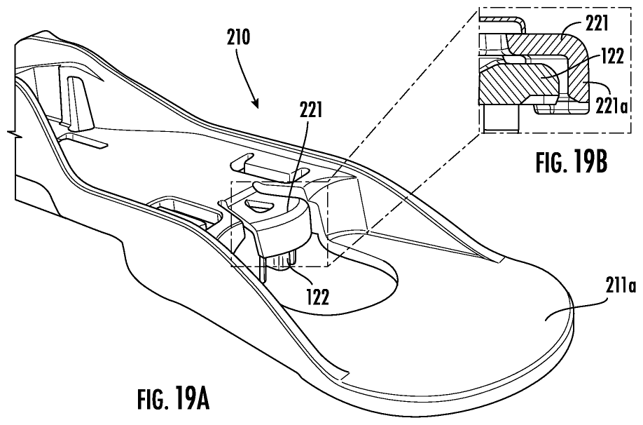

Still further, in addition to lateral, vertical and rotational alignment features, it is also contemplated that the staple cartridge insertion device may include still other alignment features. For example, in embodiments, the staple cartridge insertion device 210 may include longitudinal alignment features in order to help ensure that the staple cartridge 122 is longitudinally aligned with the staple cartridge insertion device (and with the jaw 112) and prevented from moving longitudinally relative to the staple cartridge insertion device as the staple cartridge insertion device is being inserted into its corresponding cavity within the jaw 112. For example, as shown in FIGS. 19A and 19B, the staple cartridge insertion device 210 may include longitudinal alignment element 221 in the form of a downward-facing lip 221a or other shaped structure that extends laterally downwardly from an underside surface of the main body 211 of the staple cartridge insertion device 210. In this embodiment, the longitudinal alignment element 221 engages the distal-most edge of the staple cartridge 122 so as to help prevent the staple cartridge 122 from sliding longitudinally, e.g., distally, as the staple cartridge insertion device 210 is inserting it into its corresponding cavity within the jaw 112.

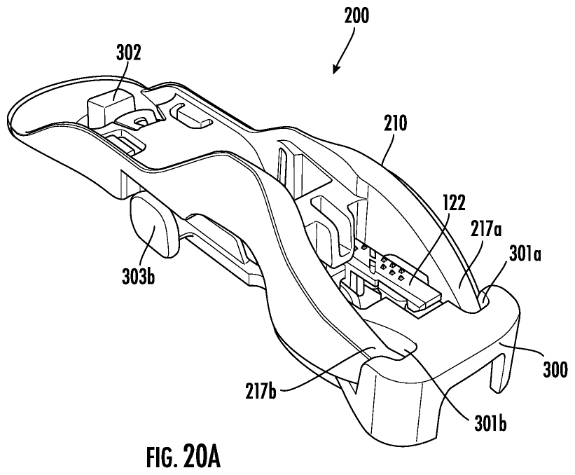

It is also contemplated that other components, in addition to a staple cartridge insertion device, may also be employed to ensure that a staple cartridge is maintained in a safe and secure state prior to its being inserted into its corresponding cavity within the jaw 112. For example, in various embodiments, the staple cartridge insertion device may also be employed with a bottom retainer element in order to protect the underside of the staple cartridge 122 (the side of the staple cartridge 122 that is opposite from the side of the staple cartridge 122 on which the staple cartridge insertion device 210 resides) prior to the staple cartridge 122 being inserted into its corresponding cavity within the jaw 112. FIG. 20A is a perspective view of a staple cartridge protection and insertion system 200 that illustrates a bottom retainer 300 that is selectively attachable and detachable to the staple cartridge insertion device 210 so as to sandwich a staple cartridge 122 therebetween and thereby protect both sides of the staple cartridge 122 prior to it being inserted into its corresponding cavity within the jaw 112.

As shown in FIGS. 20A and 20B, the bottom retainer 300 includes retainer interlock features 301a, 301b that selectively receive distal features, e.g., the proximally-extending lateral alignment wall portions 217a, 217b, of the staple cartridge insertion device 210. Another feature of the bottom retainer 300, e.g., an upward-facing nub or other protuberance 302, may be selectively insertable through an opening in the main body 211 of the staple cartridge insertion device 210 so as to help maintain, e.g., by a snap or friction fit or the like, the bottom retainer 300 and the staple cartridge insertion device 210 connected to each other (with the staple cartridge 122 disposed therebetween). When a user is ready to disconnect the bottom retainer 300 and the staple cartridge insertion device 210 from each other, the user may retract the upward-facing nub 302 of the bottom retainer 300 from out of the corresponding opening of the staple cartridge insertion device 210 and then remove the proximally-extending lateral alignment wall portions 217a, 217b, of the staple cartridge insertion device 210 from out of the retainer interlock features 301a, 301b of the bottom retainer 300. The bottom retainer 300 can then be set aside or disposed of. The staple cartridge insertion device 210 and the stapler cartridge 122, which at this time remain attached to each other, are then able to be inserted between the jaws 111, 112, until eventually the staple cartridge 122 is ejected from the staple cartridge insertion device 210 and into its corresponding cavity within the jaw 112 as previously described hereinabove.

Of course, it should be recognized that the bottom retainer 300 (and the staple cartridge insertion device 210) may have additional or different structural features that enable them to be selectively attachable and detachable to each other, or that help maintain their positions relative to each other. For example, the bottom retainer 300 may also include lateral wings 303a, 303b on opposite lateral sides thereof so as to help maintain relative lateral positioning of the bottom retainer 300 and the staple cartridge insertion device 210. Still other features are also contemplated for this purpose.

It is also contemplated that still other components, in addition to a staple cartridge insertion device and a bottom retainer, may also be employed to ensure that a staple cartridge is maintained in a safe and secure state prior to its being inserted into its corresponding cavity within the jaw 112 and to ensure that the staple cartridge is correctly inserted into its corresponding cavity in the stapler jaw. For example, in various embodiments, a staple cartridge insertion device and a bottom retainer may also be employed with a retainer guide block that interfaces with the jaw 111 in order to help ensure alignment of the staple cartridge 122 with the jaws 111, 112 prior to the staple cartridge 122 being inserted into its corresponding cavity within the jaw 112.

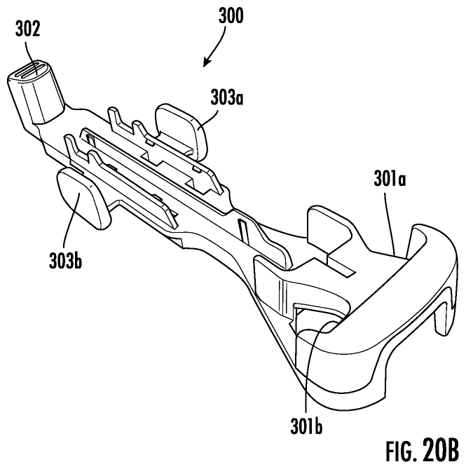

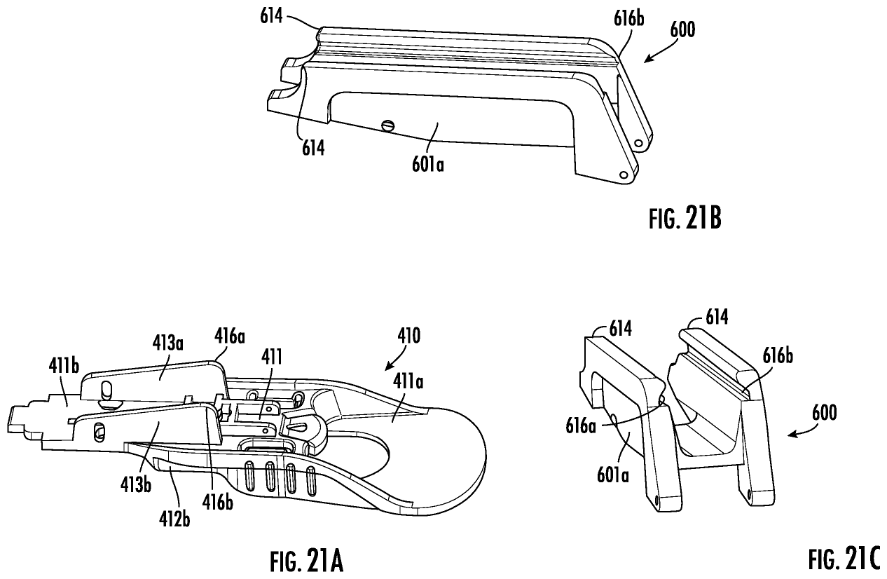

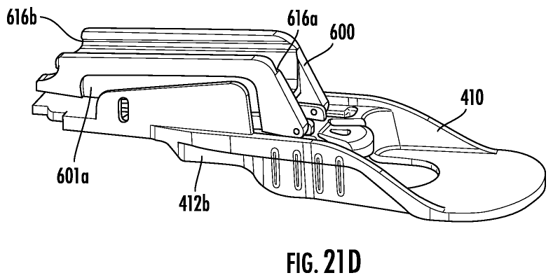

FIGS. 21A-21E are various perspective views of a staple cartridge protection and insertion system 400. In the embodiment shown, the staple cartridge protection and insertion system 400 includes a staple cartridge insertion device 410. As best shown in FIG. 21A, the staple cartridge insertion device 410 includes a main body 411 having a distal portion 411a and a proximal portion 411b. The main body 411 may include a longitudinal rib (not shown, but similar to the longitudinal rib 12 shown in FIG. 8) that removably couples or attaches the staple cartridge insertion device 410 to a staple cartridge, such as the staple cartridge 122 described above. The longitudinal rib may extend downwardly from a lower surface of the main body 411, and may be sized and shaped so as to be removably insertable into the central channel 119 of the staple cartridge 122. It should be recognized that other structures for removably coupling the staple cartridge insertion device 410 to the staple cartridge 122 are contemplated, and that a longitudinal rib similar to that shown and described in FIG. 8 is merely one example structure.

The staple cartridge insertion device 410 also includes two upwardly-extending lateral alignment walls, specifically upwardly-extending lateral alignment walls 413a and 413b, respectively. As best shown in FIG. 21D, the upwardly-extending lateral alignment walls 413a, 413b extend upwardly from the main body 411, and are sized and shaped so as to extend along and engage lateral recesses 601a, 601b (hidden) on the retainer guide block 600.

The staple cartridge insertion device 410 may also include additional lateral alignment walls. In the embodiment shown in FIGS. 21A-21E, the staple cartridge insertion device 410 includes two downwardly-extending lateral alignment walls or ribs, specifically downwardly-extending lateral alignment walls 412a and 412b, respectively. As shown, the downwardly-extending lateral alignment walls 412a, 412b extend downwardly from a proximal portion 411b of the main body 411. In addition, the downwardly-extending lateral alignment walls 412a, 412b are sized and shaped such that, when the longitudinal rib (such as shown in FIG. 8) is inserted into the central channel 119 of the staple cartridge 122, the downwardly-extending lateral alignment walls 412a, 412b extend along the outer side walls of the jaw 112 of the surgical stapler 100 that receives the staple cartridge 122.

Unlike the previous embodiments in which the staple cartridge insertion device itself includes vertical alignment features in order to ensure that the staple cartridge 122 is inserted into its corresponding cavity within the jaw 112 at an appropriate relative vertical position, the embodiment of FIGS. 21A-21E include an arrangement in which the vertical alignment features is provided separately from the staple cartridge insertion device. Specifically, in this embodiment, the proximal-most end 411b of the staple cartridge insertion device 410 is generally flat and it is the retainer guide block 600 to which it attaches that provides the vertical alignment functionality. As shown in FIGS. 21B-21E, the retainer guide block 600 includes a vertical alignment spacer 614 located near its proximal end and that, when the retainer guide block 600 is attached to the staple cartridge insertion device 410, ensures that the staple cartridge 122 is inserted into its corresponding cavity within the jaw 112 at an appropriate relative vertical position. As previously mentioned, inserting the staple cartridge 122 into its corresponding cavity within the jaw 112 at an appropriate relative vertical position may help ensure that the switch 191 of the staple cartridge 122 does not engage incorrectly the lockout features of the jaw 112.

In embodiments, the staple cartridge insertion device 410 and the retainer guide bock 600 may also include respective distal seating features in order to help ensure that the distal end of the staple cartridge 122 is fully inserted into its corresponding cavity within the jaw 112 prior to a user operating the surgical stapler instrument 100. For example, the staple cartridge insertion device 410 may include distal seating elements 416a, 416b which, in the embodiment shown, are the topmost ridges or bumps located at the highest and most distal ends of the two upwardly-extending lateral alignment walls 413a, 413b. Likewise, the retainer guide block 600 may also include upper slots 616a, 616b. As best shown in FIGS. 21D and 21E, when the upwardly-extending lateral alignment walls 413a, 413b are fitted within the lateral recesses 601a, 601b of the retainer guide block 600, the upper slots 616a, 616b of the retainer guide block 600 provide a surface against which the jaw 111 engages, as will be shown and described in additional detail below.

FIG. 21E illustrates the staple cartridge insertion device 410, the retainer guide block 600 and a bottom retainer (such as the bottom retainer 300 shown and described hereinabove) detachably connected to each other and collectively providing an insertion and protection system for a staple cartridge 122. The staple cartridge 122 is sandwiched between the staple cartridge insertion device 410 and the bottom retainer 300, with the retainer guide block 600 detachably connected to an upper region of the staple cartridge insertion device 410.

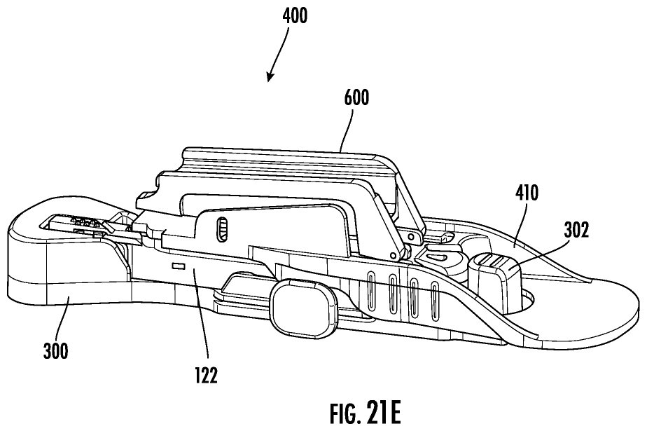

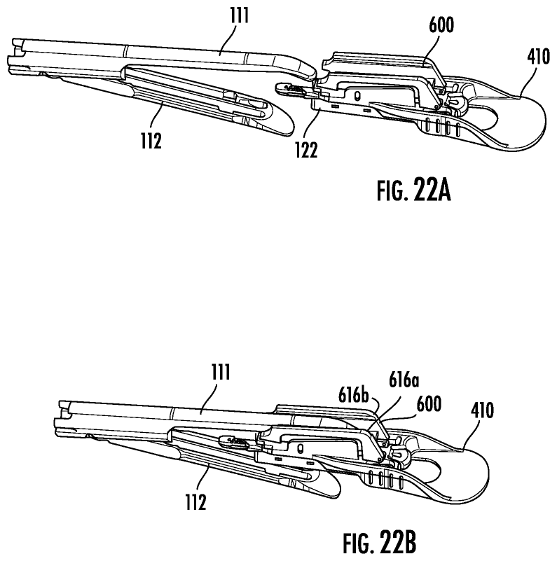

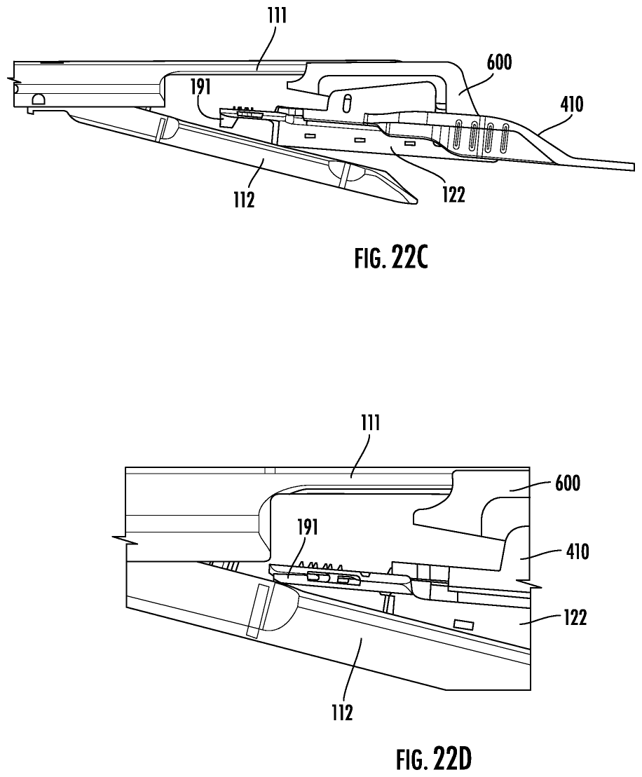

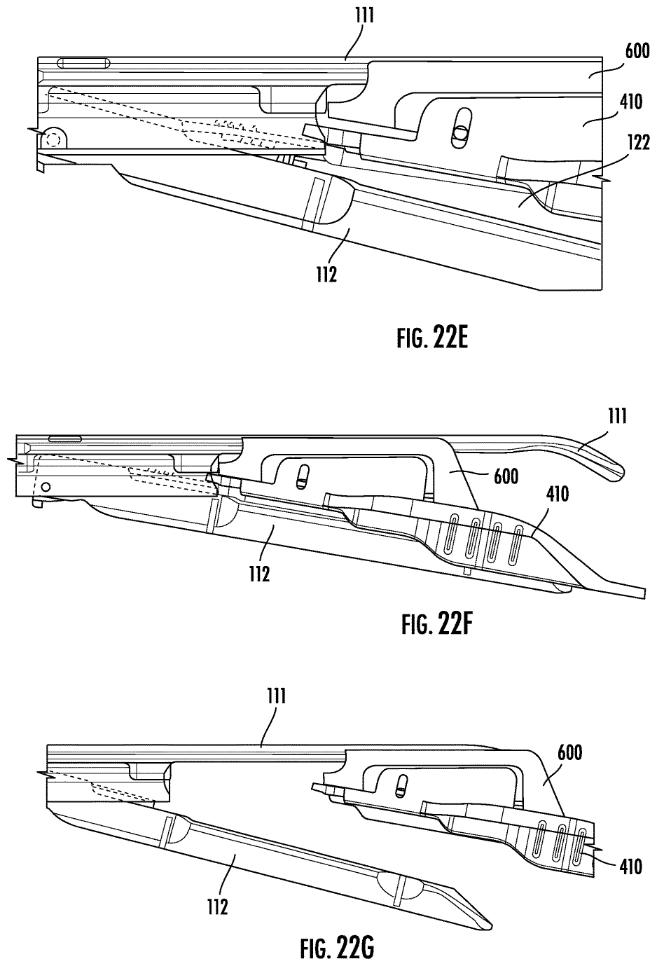

FIGS. 22A-22G illustrates the steps to employ the staple cartridge protection and insertion system 400, according to various embodiments. For example, in operation, when a staple cartridge 122 is desired to be inserted into its corresponding cavity in jaw 112, the staple cartridge 122, along with its staple cartridge protection and insertion system 400, is removed from its collective packaging (not shown). As shown in FIG. 22A, the bottom retainer 300 is removed, such as by being disconnected from the staple cartridge insertion device 410 by pressing nub 302 through its corresponding opening in the staple cartridge insertion device 410 until the bottom retainer 300 is detached therefrom. As shown in FIG. 22B, the retainer guide block 600 is moved proximally relative to the jaws 111, 112 until the tip of the jaw 111 of the staple instrument enters and slides into the upper slots 616a, 616b of the retainer guide block 600.

As shown in FIG. 22C, as the jaw 111 of the staple instrument is pushed into and slides through the upper slots 616a, 616b of the retainer guide block 600, the staple cartridge 122 engages with the jaw 112. Specifically, in the embodiment shown, a first corner of the bottom surface of the staple cartridge 122 enters into its corresponding cavity in the jaw 112. As shown in FIG. 22D, as the jaw 111 of the staple instrument continues to be slid through the upper slots 616a, 616b of the retainer guide block 600, the switch 191 of the staple cartridge 122 enters the channel slot of the jaw 112 and disables the lockout mechanism therein so that the staple cartridge 122 will be operable when subsequently fired. Once the proximal-most end of the staple cartridge 122 contacts an end point of the cavity in the jaw 112 (as seen in FIG. 22E), a user may begin to squeeze the distal ends of the jaws 111, 112 together. In this manner, the jaw 111 transfers a force to the upper slots 616a, 616b of the retainer guide block 600, which in turn transfers a force to the distal seating elements 416a, 416b of the staple cartridge insertion device 410, which in turn transfers a force to the staple cartridge 122, and in particular to the distal end of the staple cartridge 122 in order to fully seat the distal end of the staple cartridge 122 within the distal end of the cartridge-receiving cavity of jaw 112.

FIG. 22F illustrates the jaws 111, 112 squeezed sufficiently so as to seat the staple cartridge 122 within such cavity. As shown in FIG. 22G, the staple cartridge insertion device 410 and the retainer guide block 600 may then collectively be moved distally relative to the staple instrument by sliding the jaw 111 proximally through the upper slots 616a, 616b of the retainer guide block 600 until the staple cartridge insertion device 410 and the retainer guide block 600 are free to be removed from the jaw 111. The staple cartridge 122 is now able to be employed by a surgical instrument in a subsequent surgical procedure.

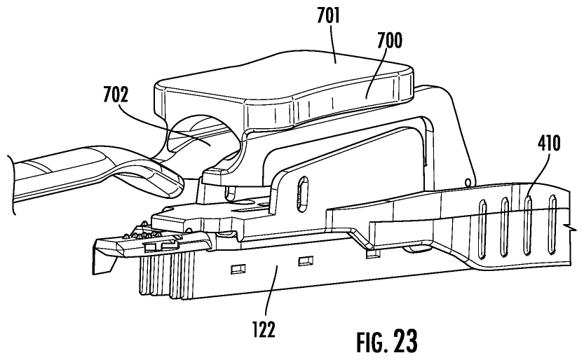

Of course, it should be recognized that the retainer guide block may come in various different shapes. For example, while the retainer guide block 600 shown and described hereinabove includes upper slots 616a, 616b for engaging the jaw 111 while having the upper surface of the retainer guide block be open, FIG. 23 illustrates an alternative embodiment in which the retainer block 700 has an upper surface 701 that is closed. In this embodiment, the retainer guide block 700 has a channel or lumen 702 for receiving the jaw 111, the tip of the jaw 111 being insertable into the channel or lumen 702 and able to be slid distally therethrough.

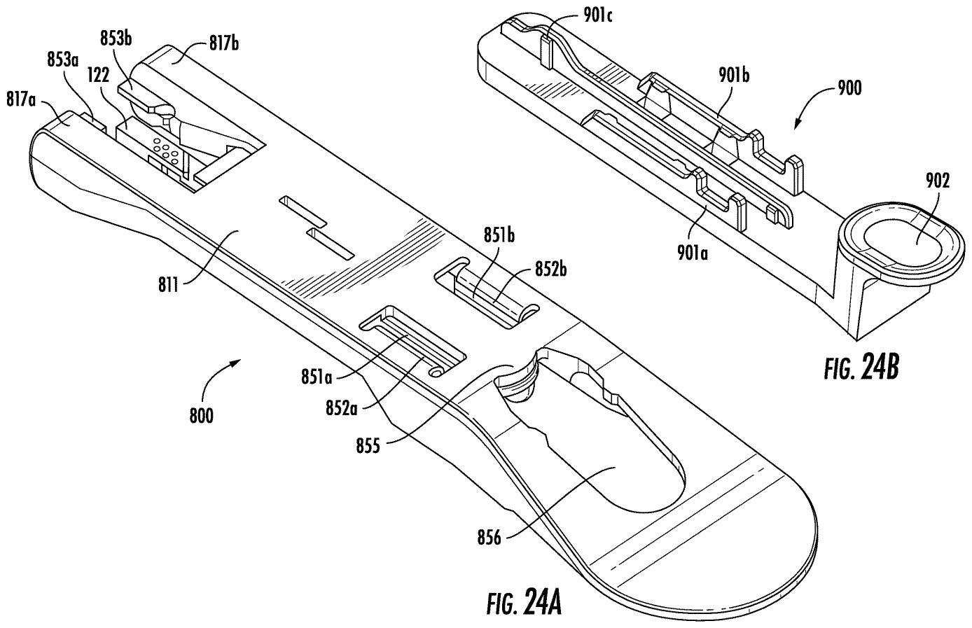

As set forth above, it is contemplated that other components, in addition to a staple cartridge insertion device, may also be employed to ensure that a staple cartridge is maintained in a safe and secure state prior to its being inserted into its corresponding cavity within the jaw 112. In various embodiments shown and described above in, e.g., FIGS. 20A and 20B, the staple cartridge insertion device may also be employed with a bottom retainer element in order to protect the underside of the staple cartridge 122 (the side of the staple cartridge 122 that is opposite from the side of the staple cartridge 122 on which the staple cartridge insertion device 210 resides) prior to the staple cartridge 122 being inserted into its corresponding cavity within the jaw 112. FIGS. 24A and 24B are perspective views of a staple cartridge protection and insertion system that illustrates another embodiment of a staple cartridge insertion device 800, as well as a bottom retainer 900 that is selectively attachable and detachable to the staple cartridge insertion device 800, so as to sandwich a staple cartridge 122 therebetween and thereby protect both sides of the staple cartridge 122 prior to it being inserted into its corresponding cartridge-receiving slot within the jaw 112.

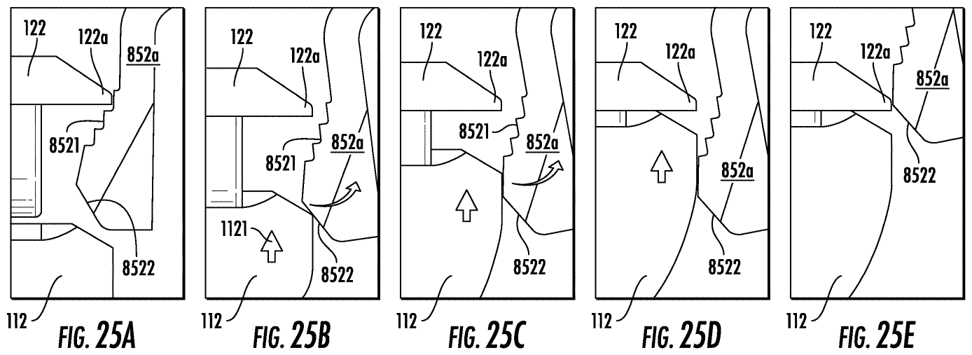

As shown in FIG. 24A, the staple cartridge insertion device 800 includes staple cartridge retention features 851a and 851b. Specifically, the staple cartridge retention features, as shown in this embodiment, include slots 851a, 851b. The slots 851a, 851b are disposed in the upper surface of the staple cartridge insertion device 800 and curve downwardly to tabs 852a and 852b, additional details of which are described below and best shown in FIGS. 25A through 25E.

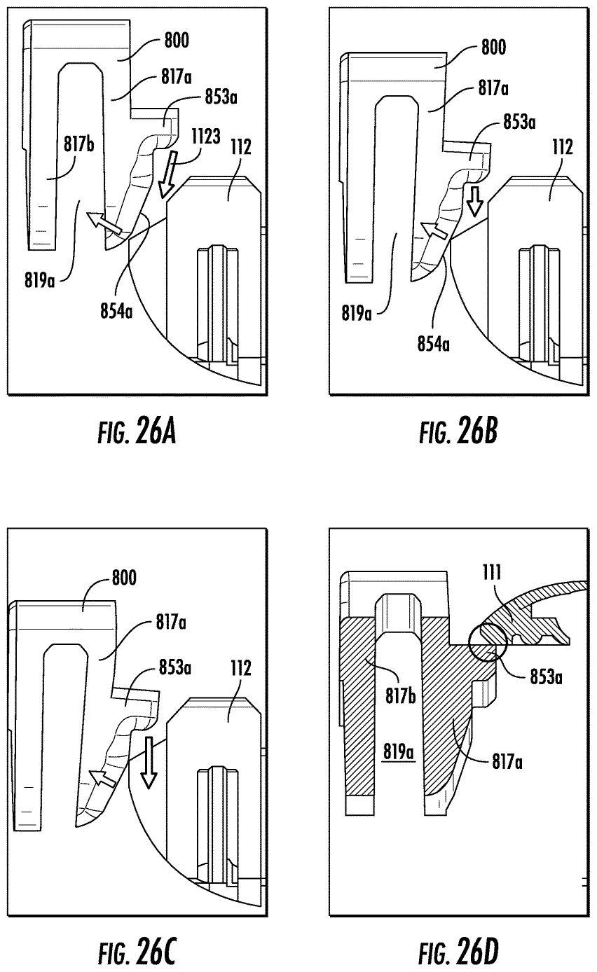

As also shown in FIG. 24A, the staple cartridge insertion device 800 may also include proximal alignment features 853a and 853b. Specifically, the proximal alignment features, as shown in this embodiment, includes protuberances 853a, 853b. The protuberances 853a, 853b extend laterally inwardly near the proximal ends of the inner lateral alignment wall portions 817a, 817b. In this embodiment, the inner lateral alignment wall portions 817a, 817b extend downwardly from the top surface 811 of the staple cartridge insertion device 800 and are generally parallel to and spaced apart from outer lateral alignment wall portions 818a, 818b, which are hidden in this view but are described below and best shown in FIGS. 26A through 26D. The inner lateral alignment wall portions 817a, 817b are separated from the outer lateral alignment wall portions 818a, 818b by channels 819a, 819b, again which are hidden in this view but are described below and best shown in FIGS. 26A through 26D. In this embodiment, the proximal alignment features 853a, 853b engage a channel of the jaw 112 during insertion of the staple cartridge 122 between the first and second jaws 111, 112 so as to help ensure that the proximal end of the staple cartridge 122 is prevented from lifting relative to the first jaw 111 during such insertion, while still allowing installation on either side of the channel.

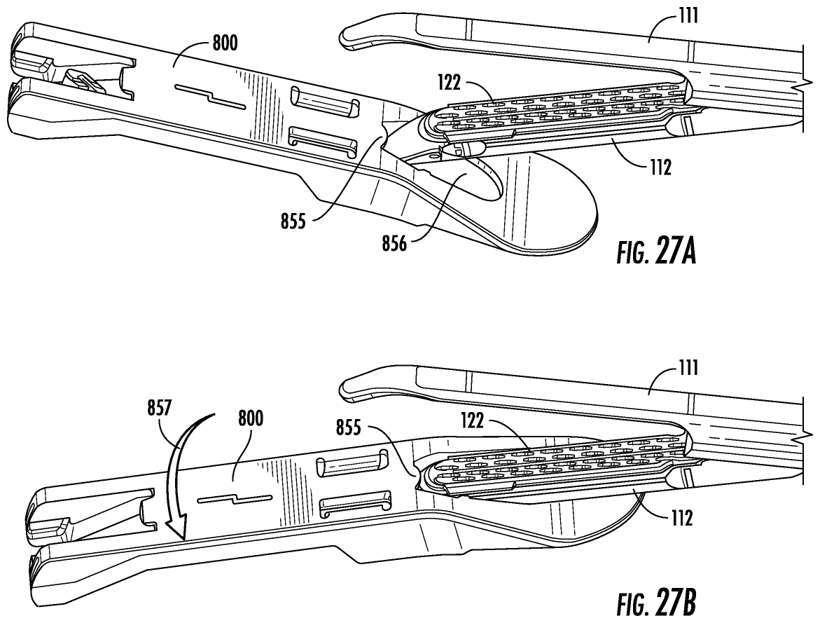

As also shown in FIG. 24A, the staple cartridge insertion device 800 may also include staple cartridge removal feature 855. Specifically, the staple cartridge removal feature 855, as shown in this embodiment, includes a staple cartridge removal lip 855. The staple cartridge removal lip 855 extends distally from the top surface 811 of the staple cartridge insertion device 800 to partially project into a void 856 defined through the top surface 811, so as to enable a loaded staple cartridge, the end of which is inserted into the void 856, to be lifted or pried out of the jaw 112, as described in additional detail below and as best shown in FIGS. 27A and 27B.

FIG. 24B, on the other hand, shows a bottom retainer 900 that is selectively attachable and detachable to the staple cartridge insertion device 800 so as to sandwich a staple cartridge 122 therebetween and thereby protect both sides of the staple cartridge 122 prior to it being inserted into its corresponding cavity within the jaw 112. As shown in FIG. 24B, the bottom retainer 900 may include retainer interlock features 901a, 901b, 901c that selectively receive complementary features of the staple cartridge insertion device 800. Another feature of the bottom retainer 900, e.g., an upward-facing nub or other protuberance 902, may be selectively insertable through the void 856 of the staple cartridge insertion device 800 so as to help maintain, e.g., by a snap or friction fit or the like, the bottom retainer 900 and the staple cartridge insertion device 800 connected to each other (with the staple cartridge 122 disposed therebetween). When a user is ready to disconnect the bottom retainer 900 and the staple cartridge insertion device 800 from each other, the user may retract the upward-facing nub 902 of the bottom retainer 900 from out of the void 856 of the staple cartridge insertion device 800 and then remove the staple cartridge insertion device 800 from out of the retainer interlock features 901a, 901b, 901c of the bottom retainer 900. The bottom retainer 900 can then be set aside or disposed of. The staple cartridge insertion device 800 and the stapler cartridge 122, which at this time remain attached to each other, are then able to be inserted between the jaws 111, 112, until eventually the staple cartridge 122 is ejected from the staple cartridge insertion device 800 and into its corresponding cavity within the jaw 112 as previously described hereinabove.

As mentioned above in connection with FIGS. 24A and 24B, the staple cartridge insertion device 800 may include slots 851a, 851b that are disposed in the upper surface of the staple cartridge insertion device 800 and curve downwardly to form tabs 852a, 852b. FIG. 25A through FIG. 25E show additional details of the tabs 852a, 852b. Referring to FIG. 25A, there is shown an exemplary tab 852a having a retention member thereon. In the example embodiment shown, the retention member is a series of steps or teeth 8521—recognizing that a single step or tooth could alternatively be employed—on a radially-inwardly facing surface thereof. As shown, the teeth 8521 engage a radially-outwardly extending lip 122a of the staple cartridge 122, thereby helping to maintain the retention of the staple cartridge 122 with the stapler cartridge insertion device 800 prior to the staple cartridge 122 and the stapler cartridge insertion device 800 being collectively inserted into the second jaw 112.

FIG. 25B shows that, as the staple cartridge 122 and the stapler cartridge insertion device 800 are collectively inserted into the second jaw 112 (such movement represented by the upward-pointing arrow 1121), the second jaw 112 engages a sloped surface 8522 of the tab 852a. The engagement of the second jaw 112 with the sloped surface 8522 of the tab 852a causes the tab 852a to move radially outwardly, thereby separating the teeth 8521 of the tab 852a from the radially-outwardly extending lip 122a of the staple cartridge 122. Continued insertion of the staple cartridge 122 and the stapler cartridge insertion device 800 into the second jaw 112, as shown in FIGS. 25C through 25E, causes the tab 852a to move fully radially outwardly until the entire tab 852a is positioned radially outwardly beyond the radially-outwardly extending lip 122a of the staple cartridge 122, allowing the stapler cartridge insertion device 800, and specifically the tab 852a thereof, to disconnect from the staple cartridge 122, while the stapler cartridge 122 is simultaneously being progressively seated within its corresponding staple cartridge-receiving slot of the second jaw 112. FIG. 25E shows the stapler cartridge 122 fully seated within its corresponding staple cartridge-receiving slot of the second jaw 112, and the tab 852a having been slid fully past the radially-outwardly extending lip 122a of the staple cartridge 122 so that the stapler cartridge insertion device 800 can be set aside or disposed.

As set forth above in connection with FIGS. 24A and 24B, the staple cartridge insertion device 800 may also include proximal alignment features 853a and 853b, e.g., protuberances 853a, 853b, along with inner lateral alignment wall portions 817a, 817b and outer lateral alignment wall portions 818a, 818b separated from each other by channels 819a, 819b. FIGS. 26A through 26D show additional details of these features. Referring to FIG. 26A, there is shown a front view of the staple cartridge insertion device 800 with the inner lateral alignment wall portion 817a and the outer lateral alignment wall portion 818a separated from each other by channel 819a. The inner lateral alignment wall portion 817a includes the protuberance 853a and a sloped surface 854a along its radially-inward surface.

FIG. 26A shows that, as the stapler cartridge insertion device 800 (along with the staple cartridge 122 that is connected thereto, but not shown in this view) is inserted into the second jaw 112 (such movement represented by the downward-pointing arrow 1123), the second jaw 112 engages the sloped surface 854a of the inner lateral alignment wall portion 817a. The engagement of the second jaw 112 with the sloped surface 854a of the inner lateral alignment wall portion 817a causes the inner lateral alignment wall portion 817a to move or flex radially outwardly, thereby helping to maintain the stapler cartridge insertion device 800 (and hence the staple cartridge 122) in alignment with the second jaw 112. The protuberance 853a prevents the staple cartridge 122 and the stapler cartridge insertion device 800 from being over-inserted into the second jaw 112, as shown in FIGS. 26B through 26C. FIG. 26D shows that the protuberance 853a also prevents the staple cartridge 122 and the stapler cartridge insertion device 800 from being lifted beyond the first jaw 111, again helping to ensure the stapler cartridge insertion device 800 (and hence the staple cartridge 122) is correctly inserted into its corresponding staple cartridge-receiving slot of the second jaw 112.

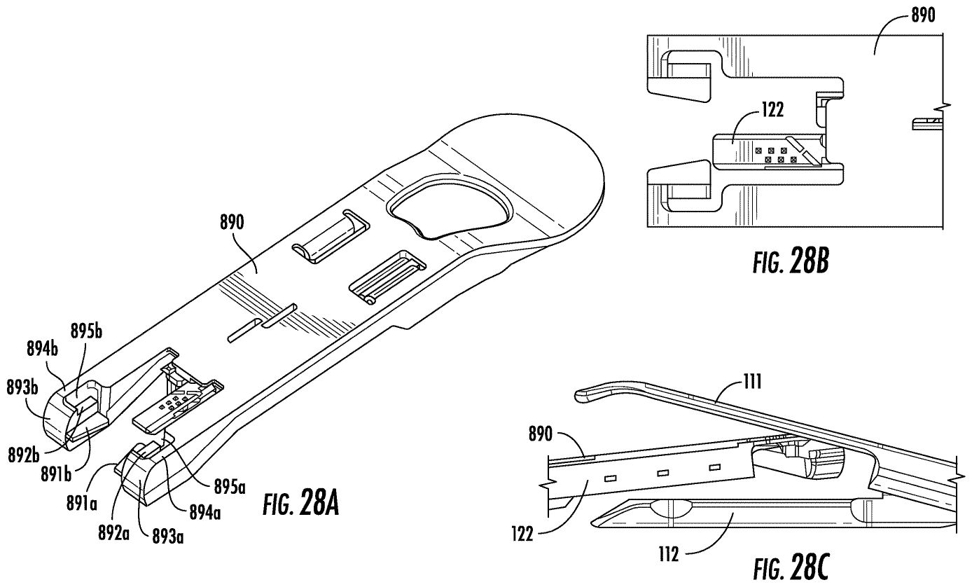

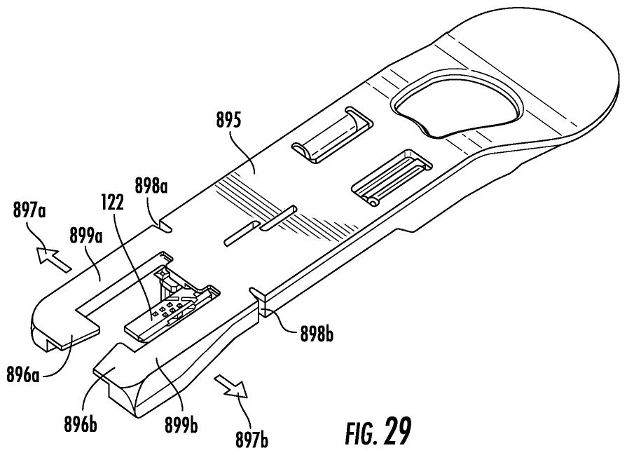

It should be noted that while FIGS. 26A through 26D show an arrangement of proximal alignment features whereby inner lateral alignment wall portions 817a, 817b are connected to a top surface of the staple cartridge insertion device 800 and that flex or move outwardly towards outer lateral alignment wall portions 818a, 818b when inserted between the jaws 111, 112, other embodiments may be employed that provide similar benefits. For example, FIGS. 28A through 28C illustrate an alternative embodiment in which protuberances 891a, 891b extend radially-inwardly from inner lateral alignment wall portions 892a, 892b. The inner lateral alignment wall portions 892a, 892b extend distally from proximal end surfaces 893a, 893b, respectively, of the staple cartridge insertion device 800, and are generally parallel to and spaced apart from outer lateral alignment walls portions 894a, 818b, respectively. The inner lateral alignment wall portions 892a, 892b are separated from the outer lateral alignment wall portions 894a, 894b by channels 895a, 895b, respectively. In this embodiment, the protuberances 891a, 891b engage the jaw 112 during insertion of the staple cartridge 122 between the first and second jaws 111, 112 so as to help ensure that the proximal end of the staple cartridge 122 is correctly aligned relative to the second jaw 112 while also being prevented from lifting relative to the first jaw 111 during such insertion (such as in shown in FIG. 28C). The engagement of the second jaw 112 with the protuberances 891a, 891b of the inner lateral alignment wall portions 892a, 892b causes the inner lateral alignment wall portions 891a, 891b to move or flex radially outwardly, thereby helping to maintain the stapler cartridge insertion device 800 (and hence the staple cartridge 122) in alignment with the second jaw 112.

As also set forth above in connection with FIG. 24A, the staple cartridge insertion device 800 may also include staple cartridge removal feature 855, e.g., a staple cartridge removal lip 855 that enables a staple cartridge 122—that is already loaded into its corresponding staple cartridge receiving slot of the second jaw 112—to be lifted or pried out of the jaw 112 so that a new stapler cartridge 122 can be loaded in its place for a subsequent staple firing. FIG. 27A shows a staple cartridge 122 already loaded into its corresponding staple cartridge receiving slot of the second jaw 112, the distal end of such second jaw 112 being inserted into the void 856 of the staple cartridge insertion device 800. Once the distal end of the second jaw 112 is sufficiently inserted into the void 856 of the staple cartridge insertion device 800 such that the distal end of the staple cartridge 122 meets the staple cartridge removal lip 855 of the staple cartridge insertion device 800, the distal end of the staple cartridge insertion device 800 is rotated downwardly—as shown by arrow 857 in FIG. 27B—such that the staple cartridge removal lip 855 lifts or pries the previously-loaded staple cartridge out of the jaw 112. In this way, another staple cartridge insertion device 800, having a second staple cartridge 122 mounted thereto, can be loaded in the jaw 112 for a subsequent staple firing.

It should also be noted that, while FIGS. 24 through 28 illustrate embodiments in which protuberances 853a, 853b and 891a, 891b extend radially-inwardly from inner lateral alignment wall portions 817a, 817b and 892a, 892b, respectively, at a location that is at a generally vertical midpoint of such wall portions, other embodiments are envisioned in which the protuberances extend radially-inwardly from wall portions at a location that is not at the generally vertical midpoint of such wall portions. Indeed, the protuberances may extend radially-inwardly from any location vertical of such wall portions. For example, FIG. 29 illustrates another embodiment of a staple cartridge insertion device 895 in which protuberances 896a, 896b extend radially-inwardly from lateral wall portions 899a, 899b, and are generally flush with a top surface of the staple cartridge insertion device 895. The staple cartridge insertion device 895 may also include one or more cutouts or reliefs, e.g., recesses 896a, 896b, that enable the lateral wall portions 899a, 899b to move or flex radially outwardly as shown by arrows 897a, 897b, thereby helping to maintain the stapler cartridge insertion device 895 (and hence the staple cartridge 122) in alignment with the second jaw 112. As described above in connection with other protuberances, in this embodiment, the protuberances 896a, 896b engage the jaw 112 during insertion of the staple cartridge 122 between the first and second jaws 111, 112 so as to help ensure that the proximal end of the staple cartridge 122 is correctly aligned relative to the second jaw 112 while also being prevented from lifting relative to the first jaw 111 during such insertion (similar to that shown in FIG. 28C).

As set forth above, FIG. 1C illustrates a robotic surgical system in which a surgical stapler instrument, and the staple cartridge insertion device 10, may be employed. FIG. 1C illustrates, as an example, a top view of an operating room employing a robotic surgical system. The robotic surgical system in this case is a robotic surgical system 300 including a Console (“C”) utilized by a Surgeon (“S”) while performing a minimally invasive diagnostic or surgical procedure, usually with assistance from one or more Assistants (“A”), on a Patient (“P”) who is lying down on an Operating table (“O”).

The Console includes a monitor 304 for displaying an image of a surgical site to the Surgeon, left and right manipulatable control devices 308 and 309, a foot pedal 305, and a processor 302. The control devices 308 and 309 may include any one or more of a variety of input devices such as joysticks, gloves, trigger-guns, hand-operated controllers, or the like. The processor 302 may be a dedicated computer that may be integrated into the Console or positioned next to it.

The Surgeon performs, e.g., a minimally invasive surgical procedure, by manipulating the control devices 308 and 309 so that the processor 302 causes their respectively associated robotic arm assemblies 328 and 329 to manipulate their respective removably coupled surgical instruments 100a and 100b accordingly, while the Surgeon views the surgical site in 3-D on the Console monitor 304 as it is captured by a stereoscopic endoscope 340.

Each of the surgical instruments 100a, 100b, as well as the endoscope 340, may be inserted through a cannula or other tool guide (not shown) into the Patient so as to extend down to the surgical site through corresponding minimally invasive incisions 366. Each of the robotic arms is conventionally formed of links, such as link 362, which are coupled together and manipulated through motor controlled or active joints, such as joint 363.

The number of surgical instruments used at one time, and consequently the number of robotic arms being used in the system 300, will generally depend on the diagnostic or surgical procedure and the space constraints within the operating room, among other factors. If it is necessary to change one or more of the surgical instruments 100a, 100b being used during a procedure, the Assistant may remove the surgical instrument no longer being used from its robotic arm, and replace it with another surgical instrument 331 (and/or a replacement staple cartridge 100 as will described in additional detail below) from a Tray (“T”) in the operating room.

The monitor 304 may be positioned near the Surgeon's hands so that it will display a projected image that is oriented so that the Surgeon feels that he or she is actually looking directly down onto the operating site. To that end, images of the surgical instruments 100a, 100b may appear to be located substantially where the Surgeon's hands are located.

The processor 302 may perform various functions in the system 300. One function that it may perform is to translate and transfer the mechanical motion of control devices 308 and 309 to their respective robotic arms 328 and 329 through control signals over a bus 310 so that the Surgeon can effectively manipulate their respective surgical instruments 100a, 100b. Another function that the processor 302 may perform is implementing various control system processes as described herein.

Although described as a processor, it is to be appreciated that the processor 302 may be implemented in practice by any combination of hardware, software and firmware. Also, its functions as described herein may be performed by one unit, or divided up among different components, each of which may be implemented in turn by any combination of hardware, software and firmware. Additional details on robotic surgical systems, can be found in, e.g., commonly owned U.S. Pat. Nos. 6,493,608, 6,671, and International Application WO 2017/132611, the entire disclosures of each being fully incorporated by reference herein.

While several embodiments have been shown in the drawings, it is not intended that the disclosure be limited thereto, as it is intended that the disclosure be as broad in scope as the art will allow and that the specification be read likewise. For example, the device described herein is not limited to only the mechanisms described herein, as other suitable devices or mechanisms are also contemplated. Therefore, the above description should not be construed as limiting, but merely as exemplifications of various embodiments. Thus, the scope of the embodiments should be determined by the appended claims and their legal equivalents, rather than by the examples given.

Persons skilled in the art will understand that the devices and methods specifically described herein and illustrated in the accompanying drawings are non-limiting exemplary embodiments. The features illustrated or described in connection with one exemplary embodiment may be combined with the features of other embodiments. Various alternatives and modifications can be devised by those skilled in the art without departing therefrom. Accordingly, it is intended to embrace all such alternatives, modifications and variances. As well, one skilled in the art will appreciate further features and advantages based on the above-described embodiments. Accordingly, it is not intended to be limited by what has been particularly shown and described, except as indicated by the appended claims.

1. A device for inserting a staple cartridge into a surgical stapler instrument having a first jaw that includes an anvil and a second jaw including a cavity for receiving the staple cartridge, at least one of the first and second jaws movable between open and closed positions, the device comprising:
a main body sized for positioning between the first and second jaws of the stapler instrument when the first and second jaws are in the open position, the main body having a coupling element for removably attaching the main body to the staple cartridge; and
a vertical alignment feature on a proximal portion of the main body, the vertical alignment feature configured to engage the proximal end of the staple cartridge such that the proximal portion of the main body is inserted at a first orientation into a proximal end of its receiving cavity in the second jaw.

2. The device of claim 1, further comprising:
at least one lateral alignment feature extending from the main body and at least one rotational alignment feature configured to engage at least one of the first and second jaws and configured to substantially rotationally align the staple cartridge with the first and second jaws during insertion of the staple cartridge.

3. The device of claim 2, wherein the at least one lateral alignment feature includes a pair of lateral alignment wall portions extending proximally from the main body and configured to engage outer side walls of the second jaw during insertion.

4. The device of claim 3, wherein the at least one rotational alignment feature includes a pair of protuberances, each protuberance extending from one of the pair of lateral alignment wall portions.

5. The device of claim 1, wherein each protuberance extends laterally inwardly from one of the pair of lateral alignment wall portions.

6. The device of claim 1, further comprising:
third and fourth lateral alignment walls extending from the main body and configured to engage outer side walls of the first jaw during insertion.

7. The device of claim 1, wherein the vertical alignment feature includes a spring structure located at a proximal end of the device, the spring structure biasing the proximal end of the staple cartridge downwardly during insertion.

8. The device of claim 1, wherein the staple cartridge includes a switch extending downwardly from a proximal portion of the staple cartridge, and the second jaw includes a locking member extending in a substantially longitudinal direction, and wherein the first orientation includes an orientation at which the vertical alignment feature ensures that the switch correctly engages the lockout upon insertion.

9. The device of claim 1, further comprising a distal seating element configured to contact the first jaw as the first jaw is moved by a user squeezing the first and second jaws towards each other from the open position to the closed position to thereby transfer force from the first jaw to a distal end portion of the staple cartridge.

10. The device of claim 1, further comprising:
a bottom retainer configured to protect an underside surface of the staple cartridge, the bottom retainer being selectively attachable and detachable from the device such that, when attached, the staple cartridge is disposed between the device and the bottom retainer, and when detached, the staple cartridge and the device remain attached to each other and are suitable to be inserted between the first and second jaw.

11. A device for inserting a staple cartridge into a surgical stapler instrument having a first jaw that includes an anvil and a second jaw that includes a cavity for receiving the staple cartridge, at least one of the first and second jaws movable between open and closed positions, the device comprising:
an insertion component that includes a main body sized for positioning between the first and second jaws of the stapler instrument when the first and second jaws are in the open position, and a vertical alignment feature on a proximal end portion of the main body, the vertical alignment feature defining a surface along which the insertion component may slide along the first jaw of the stapling instrument as the insertion component and the staple cartridge are inserted between the first and second jaws; and
a bottom retainer configured to protect an underside surface of the staple cartridge, the bottom retainer being selectively attachable to and detachable from the insertion component such that, when the bottom retainer is attached to the insertion component, the staple cartridge is disposed between the insertion component and the bottom retainer, and when the bottom retainer is detached from the insertion component, the staple cartridge and the insertion component remain attached to each other and are suitable to be inserted between the first and second jaw.

12. The device of claim 11, wherein the main body includes a coupling element for removably attaching the main body to the staple cartridge.

13. The device of claim 11, further comprising at least one lateral alignment feature configured to engage at least one of the first and second jaws and configured to substantially laterally align the staple cartridge with the first and second jaws during insertion of the staple cartridge.

14. (canceled)

15. The device of claim 11, further comprising at least one rotational alignment feature configured to engage at least one of the first and second jaws and configured to substantially rotationally align the staple cartridge with the first and second jaws during insertion of the staple cartridge.

16-17. (canceled)

18. The device of claim 11, wherein the insertion component includes a distal seating element disposed between the main body and the first jaw of the stapling instrument.

19. The device of claim 18, wherein the distal seating element is configured to contact the first jaw as the first jaw is moved from the open position to the closed position to transfer a force from the first jaw to a distal end portion of the staple cartridge.

20. The device of claim 19, wherein the force is sufficient to seat the distal end of the staple cartridge within the second jaw.

21. A device for inserting a staple cartridge into a surgical stapler instrument having a first jaw that includes an anvil and a second jaw that includes a cavity for receiving the staple cartridge, at least one of the first and second jaws movable between open and closed positions, the device comprising:
an insertion component that includes a main body sized for positioning between the first and second jaws of the stapler instrument when the first and second jaws are in the open position,
a retainer guide block selectively attachable to the insertion component, the retainer guide block including a vertical alignment feature configured to vertically align a proximal end of the staple cartridge as it is inserted into a proximal end of the cavity in the first jaw, the retainer guide block also includes a distal seating feature configured to contact the first jaw as the first jaw is moved by a user from the open position towards the closed position so as to transfer a force from the first jaw to a distal end portion of the staple cartridge sufficient to fully seat the distal end of the staple cartridge within the cavity.

22. (canceled)

23. The device of claim 21, wherein the distal seating feature includes slots configured to slidingly receive a distal tip of the first jaw.

24. The device of claim 21, wherein the distal seating feature includes a channel configured to slidingly receive a distal tip of the first jaw.

25-41. (canceled)
